(19) United States
(12) Patent Application Publication
(10) Pub. No.: US 2026/0308169 A1
(43) Pub. Date: Oct. 8, 2026

(54) Compounds Targeting STAT3 for Use in Treating Pancreatic Cancer

(71) Applicants: UNIVERSITY OF FLORIDA RESEARCH FOUNDATION, INCORPORATED, GAINESVILLE, FL (US); BOARD OF REGENTS, THE UNIVERSITY OF TEXAS SYSTEM, AUSTIN, TX (US)

(72) Inventors: Rita Ghosh, Austin, TX (US); Glenn Halff, Austin, TX (US); Kenneth L. Kho, Gainesville, FL (US); Addanki Pratap Kumar, Austin, TX (US); Danmeng Li, Gainesville, FL (US); David A. Ostrov, Gainesville, FL (US); Xiaoyu Yang, Austin, TX (US)

(21) Appl. No.: 19/630,182

(22) Filed: Mar. 26, 2026

Related U.S. Application Data
(60) Provisional application No. 63/778,641, filed on Mar. 27, 2025.

(51) Int. Cl. C07D 493/04; A61K 31/35; A61P 35/00

(52) U.S. Cl. CPC C07D 493/04; A61K 31/35; A61P 35/00

(57) ABSTRACT

Described herein are compounds that bind to the linker domain of STAT3 and have anti-pancreatic cancer activity. Also described are methods for treating pancreatic cancer in a subject by administering a therapeutically effective dose of the compound of formula (I) or (II).

### CROSS REFERENCE TO RELATED APPLICATIONS

This application claims the benefit of priority to U.S. Provisional Patent Application No. 63/778,641, filed Mar. 27, 2025, the contents of which is incorporated by reference in its entirety for all purposes.

### INTRODUCTION

Pancreatic ductal adenocarcinoma (PDAC) is projected to become the second leading cause of cancer-related deaths by 2030. Median survival for a majority of PDAC patients undergoing surgical resection is less than two years. However, fewer than 20% of patients present with resectable tumor and majority of patients are diagnosed with advanced unresectable disease.

Historically, 5-fluorouracil (5-FU), a pyrimidine analog, has been used as a single agent for the treatment of PDAC. In 1997, the US Food and Drug Administration approved gemcitabine, a nucleoside analog as a first-line therapy for advanced pancreatic cancer. In 2001, FOLFIRINOX, a combination of four drugs including folinic acid (leucovorin), 5-FU, irinotecan, and oxaliplatin, was approved for advanced pancreatic cancer. While FOLFIRINOX improved overall survival, it is associated with increased adverse events including neutropenia, diarrhea, and sensory neuropathy. The recognition of the presence of desmoplasia in PDAC led to the approval of nab-paclitaxel (albumin-bound paclitaxel). Nab-paclitaxel in combination with gemcitabine produced a modest increase in patient survival. The marginal benefit of this regimen was accompanied with adverse effects including thrombocytopenia, neutropenia, peripheral neuropathy, and pneumonitis. More recently NALIRIFOX (combination of irinotecan liposome with oxaliplatin, 5-fluorouracil and leucovorin) was approved for treatment of metastatic pancreatic cancer. NALIRIFOX significantly improved overall survival vs gemcitabine plus nab-paclitaxel in treatment naïve metastatic pancreatic cancer patients (11.1 months with NALIRIFOX vs 9.2 months with gemcitabine plus nab-paclitaxel). Despite advancements, the 5-year survival of PDAC patients still remains the lowest among the top solid tumors. Treatment regimens that can achieve improved survival and reduced toxicity are still a dire need for the management of PDAC.

It has been shown that the natural compound palmatine inhibits the growth of pancreatic cancer (PCC), pancreatic stellate cells (PSC), and PSC-PCC communication. Palmatine was also shown to potentiate gemcitabine activity in vitro and in vivo. Palmatine was predicted, by molecular docking, to preferentially bind to the linker domain of STAT3.

We now described additional compounds that bind to the linker domain of STAT3 and have anti-pancreatic cancer activity.

### SUMMARY

Described are compounds of formulas I and II, compositions containing compounds of formulas I and II and methods of treating a subject (e.g., a human subject) having pancreatic cancer using the compounds of formulas I and II and compositions containing the compounds of formulas I and II.

Described are methods of treating pancreatic cancer in a subject comprising: administering to the subject a therapeutically effective dose of a compound of formula I:

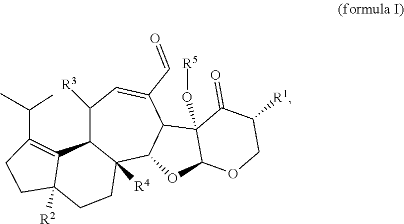

wherein:
—R¹ is

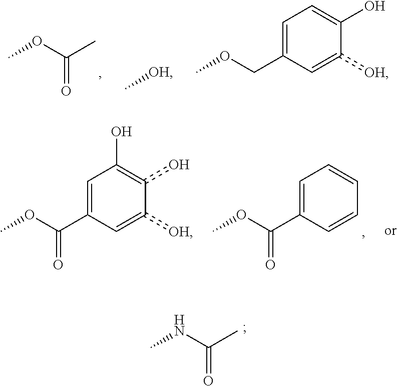

—R² is

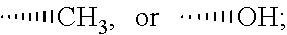

—R³ is

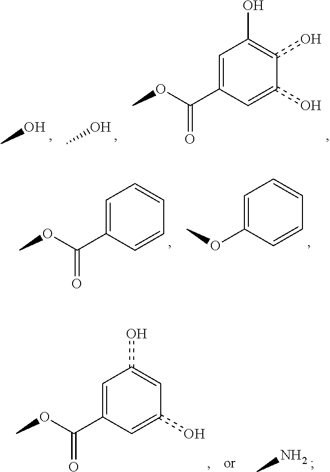

—R⁴ is —CH₃ or —OH; and
—R⁵ is —H or

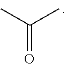

Treating pancreatic cancer can include one or more of inhibiting pancreatic cancer cell growth and/or inhibiting communication between pancreatic cancer cells and pancreatic stellate cells. The pancreatic cancer can be, but is not limited to, pancreatic ductal adenocarcinoma or metastatic pancreatic cancer. Inhibiting pancreatic cancer cell includes, but is not limited to, inhibiting the growth of pancreatic cancer cells and/or inhibiting the growth of pancreatic stellate cells. In some embodiments, the compound of formula I is Striatal B, or a compound provided in Table 1.

Communication between pancreatic cancer cells and pancreatic stellate cells includes, but is not limited to, signaling between pancreatic cancer cells and pancreatic stellate cells that leads to increased pancreatic cancer growth (proliferation), decreased apoptosis, increased cancer cell migration, increased epithelial-mesenchymal transition, stellate cell activation, increased extracellular matrix production, and/or stellate cell migration. Decreasing or inhibiting communication between pancreatic cancer cells and pancreatic stellate cells includes decreasing signal production and/or decreasing signal reception. Signals include, but are not limited to, mitogenic factors, fibrogenic factors, neurotrophic factors, paracrine stimulants, and growth factors.

In some embodiments, the compound of formula I is combined with, or formulated for use in combination with an additional cancer therapeutic. The additional cancer therapeutic can be, but is not limited to, gemcitabine, 5-fluorouracil, irinotecan, oxaliplatin, cisplatin, capecitabine, paclitaxel, nab-paclitaxel, docetaxel, or KRAS inhibitor. In some embodiments, the additional cancer therapeutic is gemcitabine. In some embodiments, the compound of formula I is Striatal B, compound 1, compound 2, compound 3, compound 4, compound 5, compound 6, compound 7, compound 8, compound 9, compound 10, compound 11, or compound 12 and the additional cancer therapeutic is gemcitabine.

In some embodiments, combinations are described, the combinations comprising a compound of formula I and an additional cancer therapeutic. The additional cancer therapeutic can be, but is not limited to, gemcitabine, 5-fluorouracil, irinotecan, oxaliplatin, cisplatin, capecitabine, paclitaxel, nab-paclitaxel, docetaxel, or KRAS inhibitor. In some embodiments, the additional cancer therapeutic is gemcitabine. In some embodiments, the compound of formula I is Striatal B, compound 1, compound 2, compound 3, compound 4, compound 5, compound 6, compound 7, compound 8, compound 9, compound 10, compound 11, or compound 12 and the additional cancer therapeutic is gemcitabine.

In some embodiments, methods are described for treating a subject having pancreatic cancer comprises administering to the subject a compound of formula I and an additional cancer therapeutic. The additional cancer therapeutic can be, but is not limited to, gemcitabine, 5-fluorouracil, irinotecan, oxaliplatin, cisplatin, capecitabine, paclitaxel, nab-paclitaxel, docetaxel, or KRAS inhibitor. In some embodiments, the additional cancer therapeutic is gemcitabine. In some embodiments, the compound of formula I is Striatal B, compound 1, compound 2, compound 3, compound 4, compound 5, compound 6, compound 7, compound 8, compound 9, compound 10, compound 11, or compound 12 and the additional cancer therapeutic is gemcitabine. The compound of formula I can be administered in combination with the additional cancer therapeutic, concurrently with the additional cancer therapeutic, prior to administration of the additional cancer therapeutic, or after administration of the additional cancer therapeutic

In some embodiments, the methods comprising administering to the subject an effective amount (e.g., a therapeutically effective amount) of compound of formula I or II or a composition containing the compound of formula I or II. Administering compound of formula I or II to a subject can optionally be combined with one or more steps of administering one or more additional (i.e., second, third, etc.) therapeutics or treatments. The compound of formula I or II and additional therapeutics can be administered in a single composition or they may be administered separately. Examples of additional therapeutics include, but are not limited to, a pancreatic cancer therapeutic known in the art. Exemplary pancreatic cancer therapeutics include, but are not limited to, gemcitabine, 5-fluorouracil, irinotecan, oxaliplatin, cisplatin, capecitabine, paclitaxel, nab-paclitaxel, docetaxel, and KRAS inhibitors. KRAS inhibitors include, but are not limited to, adagrasib and sotorasib.

Described are compounds having the structure of formula II:

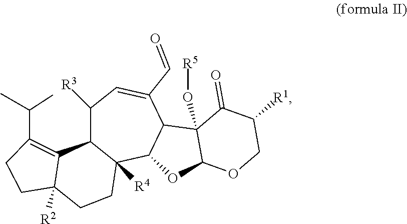

wherein:
   —R¹ is

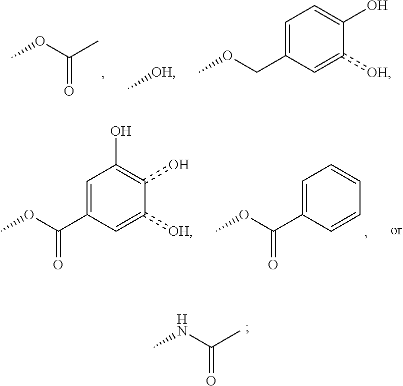

—R² is

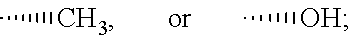

—R³ is

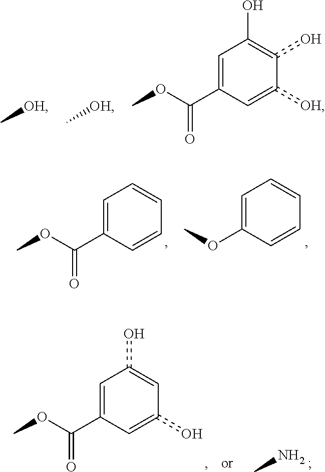

—R⁴ is —CH₃ or —OH; and
—R⁵ is —H or

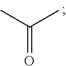

and
wherein:
   (a) if —R¹ is

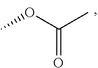

then
   —R² is

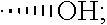

—R³

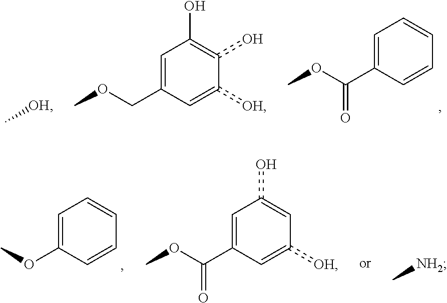

—R⁴ —OH; and/or
—R⁵

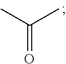

(b) if —R² is

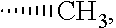

then
—R¹ is,

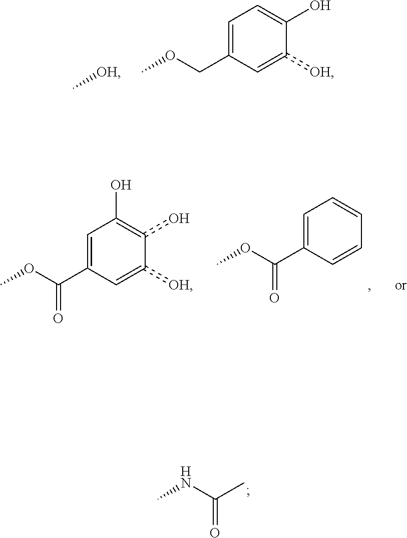

—R³ is

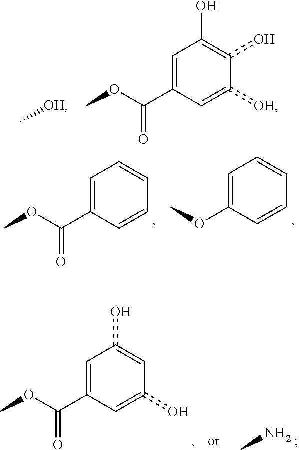

—R⁴ —OH; and/or
—R⁵ is

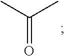

(c) if —R³ is

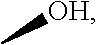

then
   —R¹ is

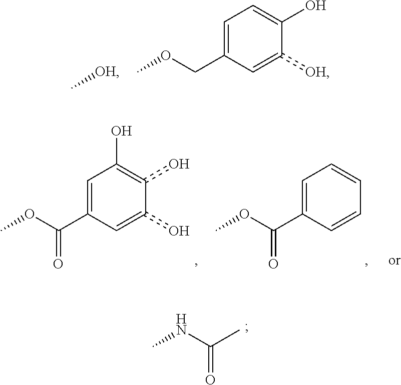

—R² is

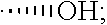

—R⁴ is —OH; and/or
—R⁵ is

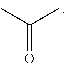

(d) if —R⁴ is —CH₃, then
   —R¹ is

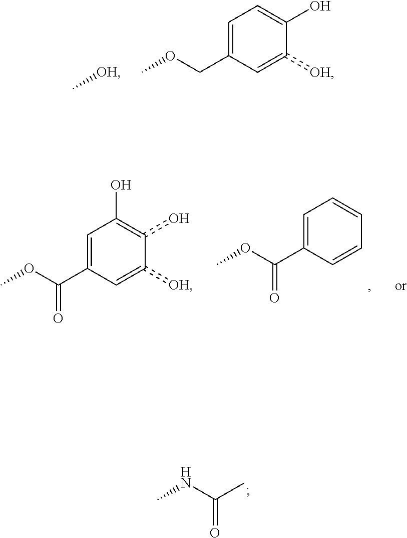

—R² is

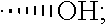

—R³ is

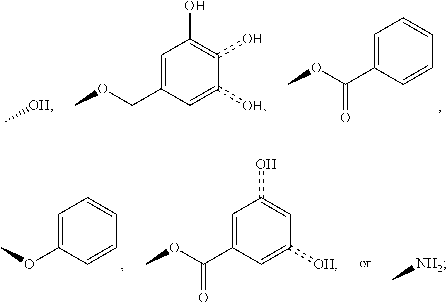

and/or
—R⁵ is

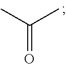

and
(e) if —R⁵ is —H; then
   —R¹ is

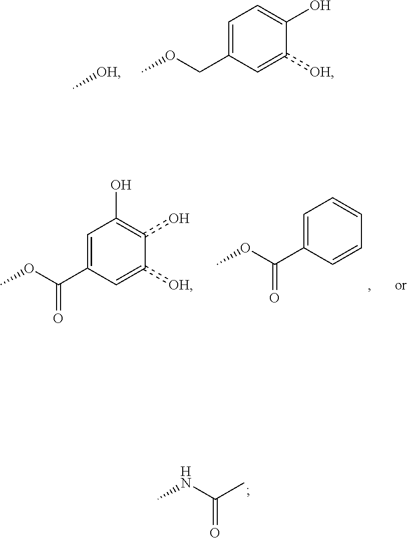

—R² is

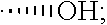

—R³ is

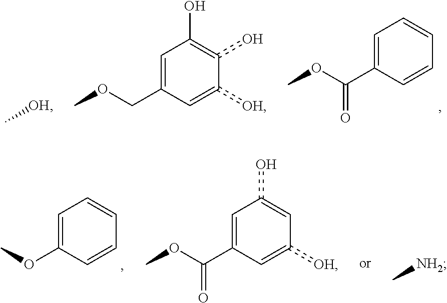

and/or
—R⁴ is —OH.

In some embodiments, a compound having the structure of formula II is a compound as provided in Table 1 (i.e., compound 1, compound 2, compound 3, compound 4, compound 5, compound 6, compound 7, compound 8, compound 9, compound 10, compound 11, or compound 12.

Also described are pharmaceutical compositions comprising any of the compounds of formula I or II. In some embodiments, a pharmaceutical composition contains a compound of formula I or II and one or more pharmaceutically acceptable excipients. In some embodiments, a pharmaceutical composition contains a compound of formula I or II and one or more additional pancreatic cancer therapeutic. In some embodiments, a pharmaceutical composition contains a compound of formula I or II, one or more additional pancreatic cancer therapeutic, and one or more pharmaceutically acceptable excipients. In some embodiments, a pharmaceutical composition containing compound of formula I or II is formulated for oral, intravascular, intravenous, intramuscular, subcutaneous, intradermal, or intraperitoneal administration. In some embodiments, a pharmaceutical composition comprises a compound of formula I or II and gemcitabine. In some embodiments, the pharmaceutical compositions are for use in treating pancreatic cancer in a subject.

Other features and advantages of the invention will be apparent from the following detailed description, and from the claims.

### BRIEF DESCRIPTION OF THE DRAWINGS

FIG. 1. Images illustrating identification of Striatal B by molecular docking. A. Ribbon diagram of full length human STAT3 predicted by AlphaFold. B and C. Molecular surface of the STAT3 linker domain. The box depicts the site where compounds were docked and outlines the boundary of the scoring grid utilized for molecular docking. Striatal B is shown as a stick model.

FIG. 2. Graph illustrating Striatal B treatment inhibits proliferation of human pancreatic cancer cell lines. PANC-1, ASPC-1, human pancreatic stellate (PSC), and mouse pancreatic cancer KPC cells were treated with increasing concentrations of Striatal B and cell proliferation was measured using MTT assay. Data presented is an average±sd of 3 independent experiments conducted in triplicate.

FIG. 3A. Graphs and images illustrating Striatal B treatment inhibits colony formation of human pancreatic cancer and mouse KPC cell lines. Logarithmically growing PANC-1 (n=3), PSC and KPC cells were treated with 2.5 μM Striatal B or vehicle control for 48 h. 500 live cells were seeded in complete media, and stained colonies were counted after 7-14 days. For ASPC-1, logarithmically growing ASPC-1 cells (n=3) were treated with 0.5 or 1 μM Striatal B or vehicle control for 48 h, 500 live cells were grown for 7-14 days until colonies were formed. Stained colonies were counted and data presented is an average±SD of three independent experiments conducted in triplicate. VC is vehicle control and Tx is Striatal B treated.

FIG. 3B. Representative crystal violate images from one experiment as described in FIG. 3A.

FIG. 4A. Images illustrating the effect of Striatal B on growth of pancreatic organoid models. Images of organoids at day 0 and following treatment with Striatal B for 5 days. IC₅₀ concentrations calculated from dose-response experiments. IC₅₀ values representing concentration required for 50% growth inhibition is shown next to each model.

FIG. 4B. Graph illustrating effect of Striatal B on growth of pancreatic organoid models. Pancreatic organoid lines were treated with increasing concentrations of Striatal B. Growth inhibition data is shown as an average±sd of 3 independent experiments conducted in triplicate is shown.

FIG. 5A. Evaluation of the combination effect of Striatal B and gemcitabine on cell viability of pancreas organoid line PA5389. PA5389 was treated with increasing concentrations of Striatal B (0-10 μM) or gemcitabine (0-400 nM) alone and in combination for 5 days and cell viability was determined. Drug response curves for both drugs and drug inhibition heatmap is shown.

FIG. 5B. Combination IC₅₀ was calculated by CrownSyn analysis. Synergy scores (Bliss independence and Loewe additivity model greater than 5 indicates synergy, and less than −5 indicates antagonism.

FIG. 6A. Single cell proteomic profiling of pancreatic cancer cells. CyTOF was performed using human pancreatic PANC-1 cells treated or Striatal B for 24 h. (A) tSNE scatter plots of subpopulations. (B) Expression heat map of signaling molecules of each subpopulation. Subpopulation changes in response to treatment are shown. Sizes of each subpopulation are indicated on the side. (C) Violin plots of indicated proteins showing response to treatment. Statistical significance calculated by student T test is shown. *p<0.05, **P<0.01, ***P<0.001.

FIG. 6B. Single cell proteomic profiling of pancreatic cancer cells. CyTOF was performed using human pancreatic ASPC-1 cells treated or Striatal B for 24 h. (A) tSNE scatter plots of subpopulations. (B) Expression heat map of signaling molecules of each subpopulation. Subpopulation changes in response to treatment are shown. Sizes of each subpopulation are indicated on the side. (C) Violin plots of indicated proteins showing response to treatment. Statistical significance calculated by student T test is shown. *p<0.05, **P<0.01, ***P<0.001.

FIG. 7. Graph illustrating differential basal epithelial markers between PDAC cell lines. Basal mRNA expression of STAT3 and genes associated with epithelial mesenchymal transition (EMT) in ASPC-1 cells relative to PANC-1 cells. Data from real time qPCR analysis presented is based on 3 independent experiments. mRNA expression normalized to β-actin and means±SD of triplicates are shown. *P<0.05, **P<0.01 relative to PANC-1 cells (VC; Student's t-test).

FIG. 8. Oncogenic signaling changes in response to treatment with Striatal B as a function of pSTAT3 levels in ASPC-1 and PANC-1 cells. Single cell proteomic profiling was performed in human pancreatic ASPC-1 and PANC-1 cells treated or Striatal B by CyTOF. A. tSNE scatter plots of subpopulations. B. Expression heat map of signaling molecules of each subpopulation and changes in response to treatment is shown. Size of each subpopulation is shown on the side. C. Violin plots of indicated proteins showing response to treatment. Statistical significance calculated by student T test is shown. *p<0.05, **P<0.01, ***P<0.001.

FIG. 9. Striatal B inhibits activated STAT3 and signaling. A. Protein levels of total and phosphorylated STAT3, and AXL in PANC-1 and ASPC-1 cells following treatment with 1 μM Striatal B for 24 h using immunoblotting. A representative image from 3 independent experiments is shown. T is Striatal B treated and VC is vehicle control. B. Binding mode of Striatal B in the binding site of STAT3.

FIG. 10. Striatal B changes the mechanical properties of PDAC cell line. Hierarchical cluster analysis followed by principal component analysis (PCA) of stiffness, adhesion and deformation revealed the significantly increased adhesion and stiffness coupled with decreased deformability following treatment (A). Violin plots display cell adhesion (nanoNewton (nN); B) and cell stiffness (Young's modulus, kPa; C) and deformation (μM; D). Data presented is mean±SD of two independent experiments. P values shown are relative to vehicle control (t-test).

### DETAILED DESCRIPTION

The scope of the present invention is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context. Additional definitions for the following and other terms are set forth throughout the specification. Patent and non-patent literature references cited within this specification, or relevant portions thereof, are incorporated herein by reference in their entireties.

### I. Definitions

The singular forms of the articles “a,” “an,” and “the” include plural references unless the context clearly dictates otherwise. For example, the term “an element” or “at least one element” can include a plurality of elements, including mixtures thereof.

The term “and/or” refers to and encompasses any and all possible combinations of one or more of the associated listed items, as well as the lack of combinations when interpreted in the alternative (“or”).

Designation of a range of values includes all integers within or defining the range, and all subranges defined by integers within the range.

“Optional” or “optionally” means that the subsequently described event or circumstance may or may not occur and that the description includes instances in which the event or circumstance occurs and instances in which the event or circumstance does not.

As used herein, the terms “about” and “approximately,” when used to modify an amount specified in a numeric value or range, indicates the numeric value as well as reasonable deviations from the value known to the skilled person in the art. In some embodiments, the term “about” means within the typical ranges of tolerances in the art. In some embodiments, the term “about” means within 1 or 2 standard deviations from the mean. In some embodiments, the term “about” means±10%. In some embodiments, the term “about” means±5%. When the term “about” is present before a series of numbers or a range, it is understood that “about” can modify each of the numbers in the series or range.

In the field of molecular modeling, “molecular docking” is a method/computer algorithm which predicts the preferred orientation of one molecule to a second when a ligand and a target are bound to each other to form a stable complex and/or strength of association or binding affinity between two molecules using. Molecular docking can be used to predict the binding-conformation of small molecule ligands to a target binding site.

A KRAS inhibitor is a drug that inhibits the oncogenic KRAS protein. KRAS is one of the most commonly mutated proteins in cancer. KRAS inhibitors include, but are not limited to, G12C inhibitors (which trap G12C mutant KRA in an inactive state), pan-KRAS inhibitors, sotorasib, adagrasib, MRTX1133, BI-2865, and RMC-6291.

The terms “treat,” “treatment,” and the like, mean the methods or steps taken to provide relief from, or amelioration or alleviation of the number, severity, adverse effect, and/or frequency of one or more symptoms or pathological consequences of a disease, disorder, or condition in a subject. Treatment can be prophylactic in terms of preventing or partially preventing a disease, or a symptom or condition of the disease. Preventing includes providing prophylaxis with respect to the occurrence or recurrence of a disease in a subject. Preventing also includes providing prophylaxis with respect to the occurrence or recurrence of a symptom or pathological consequence of a disease in a subject. Treatment can also be prophylactic in terms of delaying onset of a disease, or a symptom or condition of the disease. Delaying development of a disease or symptom or pathological consequence of the disease indicates deferring, hindering, slowing, retarding, stabilizing, suppressing, and/or postponing development of the disease or symptom or pathological consequence of the disease. The delay can be of varying lengths of time, depending on the history of the disease and/or individual being treated. Treating can include inhibiting the disease, disorder, or condition, e.g., impeding its progress; and relieving the disease, disorder, or condition, e.g., causing regression of the disease, disorder, and/or condition. Treatment can also mean prolonging survival as compared to expected survival in the absence of treatment. Treatment can be therapeutic in terms of a partial or complete cure of a disease, condition, symptom, or adverse effect attributed to the disease, disorder, or condition. The term treatment can include: (a) inhibiting the disease, i.e., arresting its development; and (b) relieving the disease, i.e., mitigating or ameliorating the disease and/or its symptoms or conditions; or (b) preventing the disease from recurring in a subject. Treating can refer to both therapeutic treatment alone, prophylactic treatment alone, or both therapeutic and prophylactic treatment. Those in need of treatment (subjects in need thereof) can include those previously diagnosed with a disease, disorder, or condition, or those identified as being at risk of developing a disease, disorder, or condition. Treating the disease, disorder, or condition can include ameliorating at least one symptom of the particular disease, disorder, or condition, even if the underlying pathophysiology is not affected.

An “effective amount” of an agent, e.g., a compound of formula I or II or a pharmaceutical composition containing a compound of formula I or II, in the context of administration, refers to an amount effective, at dosages/amounts and for periods of time necessary, to achieve a desired result, such as a therapeutic result.

A “therapeutically effective amount” of an agent, e.g., a compound of formula I or II or a pharmaceutical composition containing a compound of formula I or II, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

A “dose,” “unit dose,” or “dosage” refers to physically discrete units suitable for use in a subject, each unit containing a predetermined quantity of active pharmaceutical ingredient and/or a pharmaceutical composition.

A “subject” refers to an animal that is the object of treatment, observation, or experiment. “Animal” includes cold- and warm-blooded vertebrates and invertebrates such as fish, shellfish, reptiles and, in particular, mammals. In some embodiments, the subject is a mammal. Mammal includes, but is not limited to, mice, rats, rabbits, guinea pigs, dogs, cats, sheep, goats, cows, horses, primates, such as monkeys, chimpanzees, and apes, and humans. In some embodiments, the subject is a human.

### II. Pancreatic Cancer Therapeutics

Described are compounds for treating pancreatic cancer. The compounds include compounds of formula I and formula II, or variants thereof having affinity for the linker domain of STAT3 (amino acids about 466 to about 583) greater than or equal to that of Striatal B. In some embodiments, affinity to the linker domain of STAT3 is determined by molecular docking.

Using exploratory studies (i) tumor markers associated with epithelial mesenchymal transition (EMT) and (ii) mechanical properties were assessed using CyTOF and atomic force microscopy (AFM) respectively. This screen identified Striatal B as the top hit compound. Additional analyses demonstrated that Striatal B treatment decreased levels of STAT3 and EMT markers. Computational modeling (molecular docking) studies showed Striatal B binds to STAT3 (e.g., the linker domain of STAT3). Based on the binding of Striatal B to STAT3, compounds related to Striatal B, i.e., compounds of formulas I and II were made.

Striatal B (CHEMBL1212998) is a diterpenoid reported in Hericium coralloides and Cyathus striatus. Striatal B has a structure represented by:

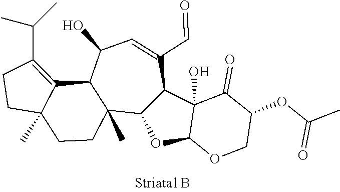 [SMILES: CC(=O)O[C@@H]1CO[C@H]2O[C@H]3[C@@H](C(C=O)=C[C@H](O)[C@@H]4C5=C(C(C)C)CC[C@]5(C)CC[C@@]34C)[C@@]2(O)C1=O]

[(1R,2S,4S,7R,9S,10R,13S,14R,19R)-11-formyl-9,13-dihydroxy-1,19-dimethyl-8-oxo-16-propan-2-yl-3,5-dioxapentacyclo[12.7.0.02,10.04,9.015,19]henicosa-11,15-dien-7-yl]acetate

In some embodiments, compounds that bind STAT3 linker domain with affinity equal to or greater than Striatal B are described.

In some embodiments, compounds that bind STAT3 linker domain with affinity equal to or greater than Striatal B comprise compounds of formula I,

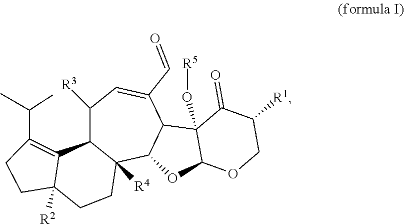

wherein:
   —R¹ is

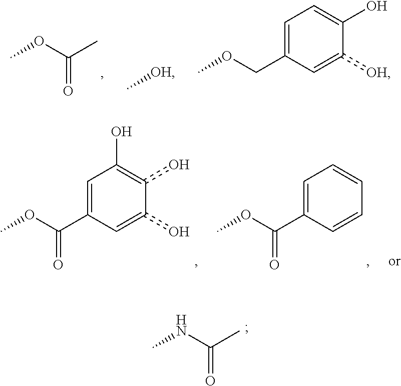

—R² is

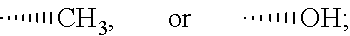

—R³ is

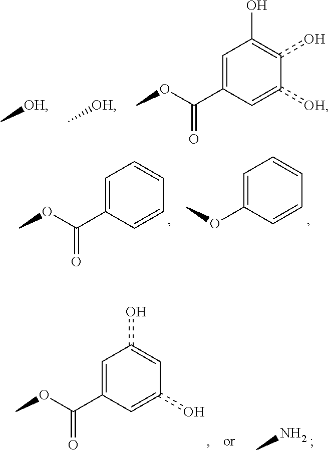

—R⁴ is —CH₃ or —OH; and
—R⁵ is —H or

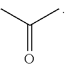

In some embodiments, the compound of formula I is not Striatal B.

In some embodiments, compounds that bind STAT3 linker domain with affinity equal to or greater than Striatal B comprise compounds of formula II,

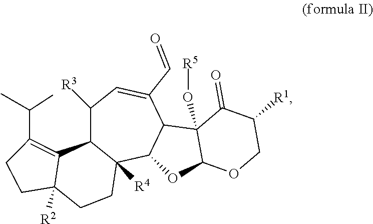

wherein:
   —R¹ is

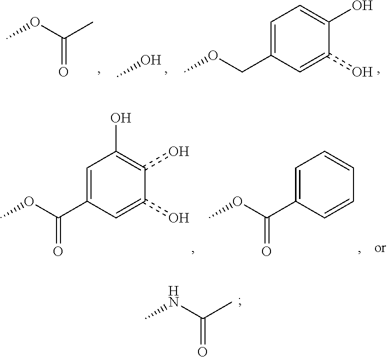

—R² is

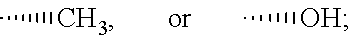

—R³ is

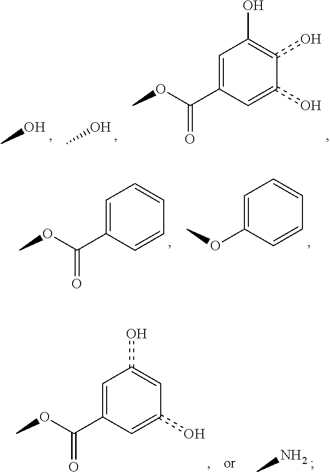

—R⁴ is —CH₃ or —OH; and
—R⁵ is —H or

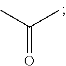

and
wherein:
   (a) if —R¹ is

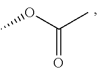

then
   —R² is

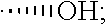

—R³

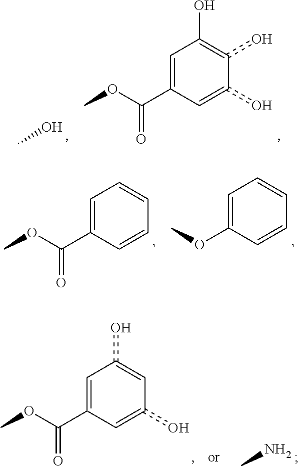

—R⁴ —OH; and/or
—R⁵

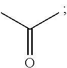

(b) if —R² is

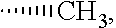

then
—R¹ is

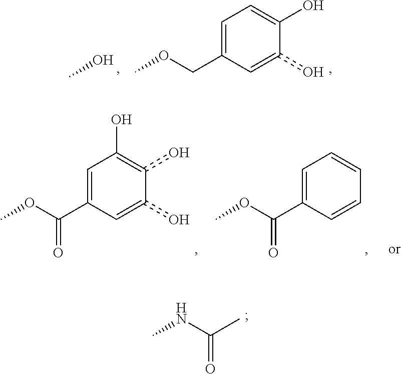

—R³ is

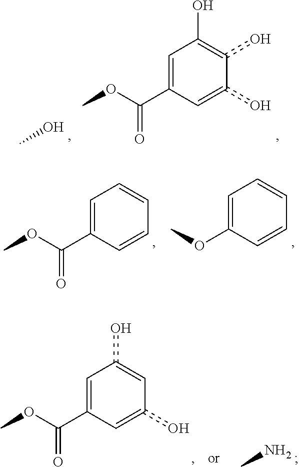

—R⁴ —OH; and/or
—R⁵ is

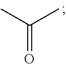

(c) if —R³ is

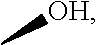

then
   —R¹ is

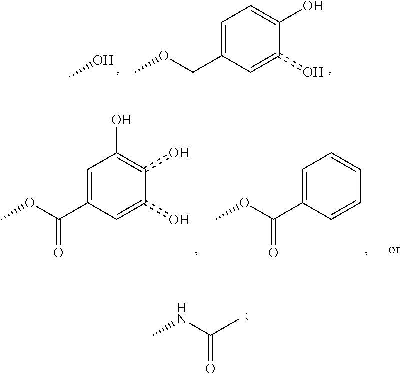

—R² is

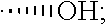

—R⁴ is —OH; and/or
—R⁵ is

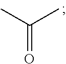

(d) if —R⁴ is —CH₃, then
   —R¹ is

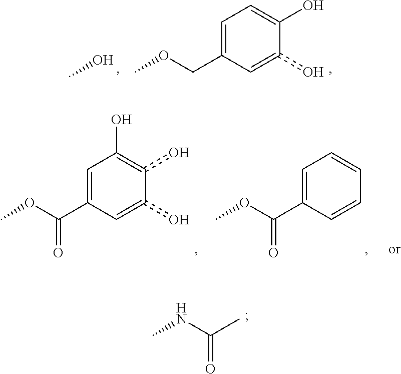

—R² is

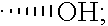

—R³ is

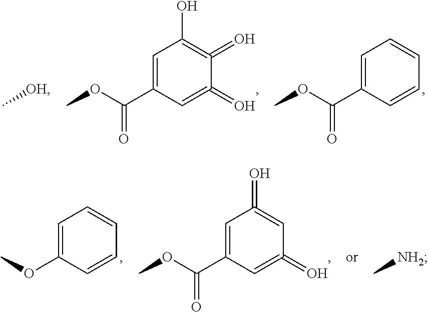

and/or
—R⁵ is

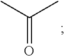

and
(e) if —R⁵ is —H; then
   —R¹ is

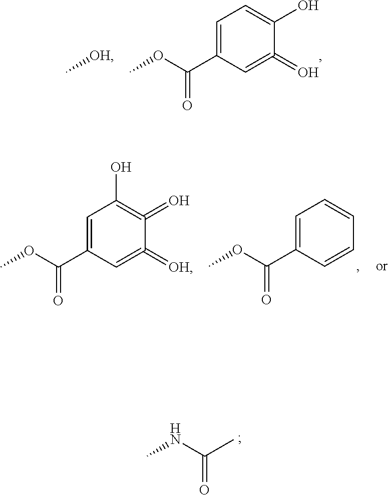

—R² is

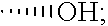

—R³ is

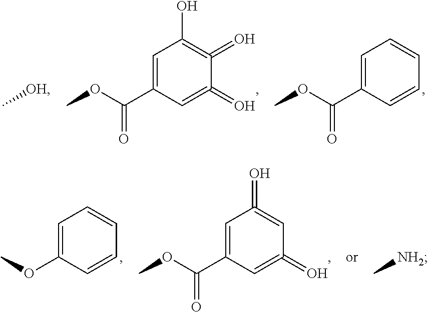

and/or
—R⁴ is —OH.

In some embodiments, —R¹ is:

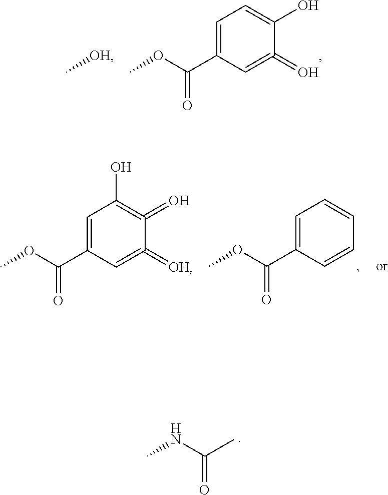

In some embodiments, —R³ is:

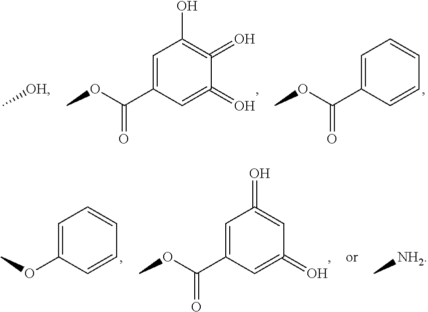

In some embodiments, —R⁴ is —OH.

In some embodiments, —R⁵ is

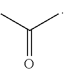

In some embodiments, —R¹ is

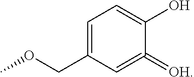

In some embodiments, —R¹ is

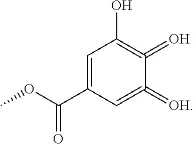

In some embodiments, —R¹ is

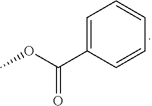

In some embodiments, —R² is

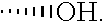

In some embodiments, —R³ is

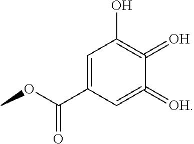

In some embodiments, —R³ is

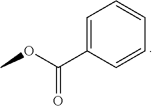

In some embodiments, —R⁴ is —OH.

In some embodiments,
—R¹ is

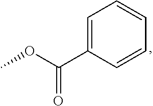

—R³ is

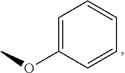

and —R⁵ is

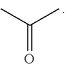

In some embodiments, —R¹ is:

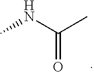

—R¹ is

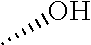

and —R³ is

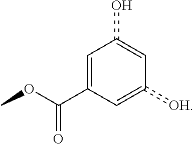

In some embodiments, —R³ is

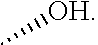

In some embodiments, —R³ is

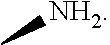

In some embodiments, compounds that bind STAT3 linker domain with affinity equal to or greater than Striatal B comprise compounds of formula II,

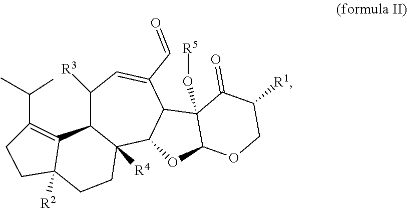

wherein
   (a) —R¹ is

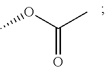

—R² is

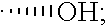

—R³

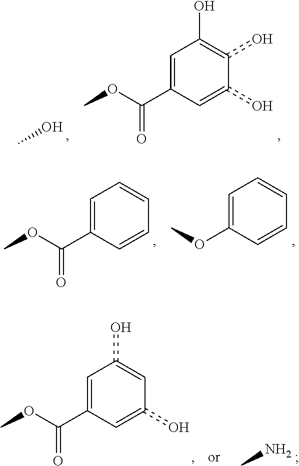

—R⁴ —OH; and
—R⁵

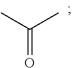

(b) —R¹ is

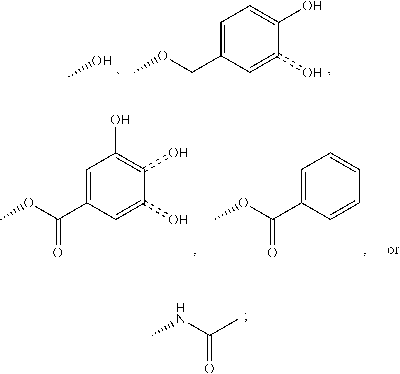

—R² is

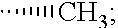

—R³ is

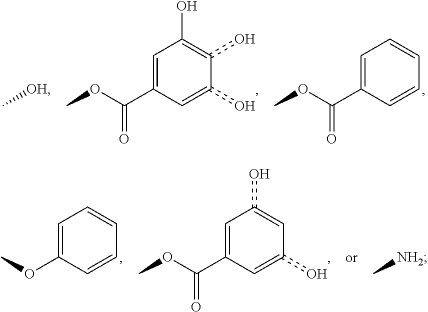

—R⁴ —OH; and
—R⁵ is

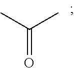

(c) —R¹ is

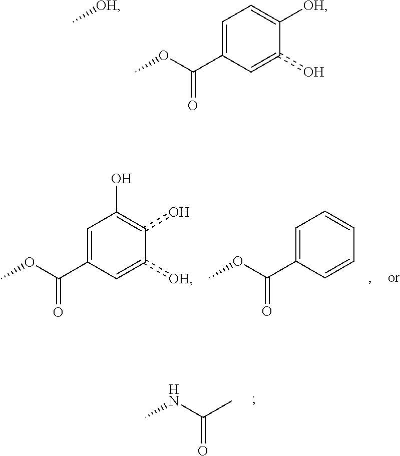

—R² is

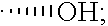

—R³ is

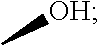

—R⁴ is —OH; and
—R⁵ is

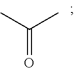

(d) —R¹ is

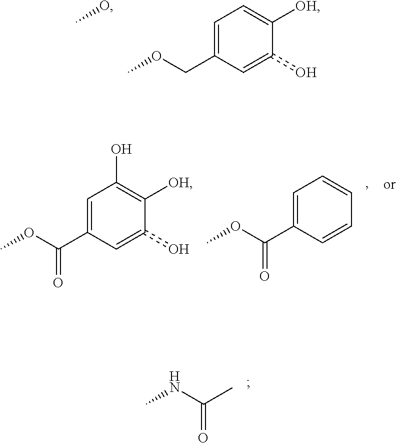

—R² is

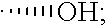

—R³ is

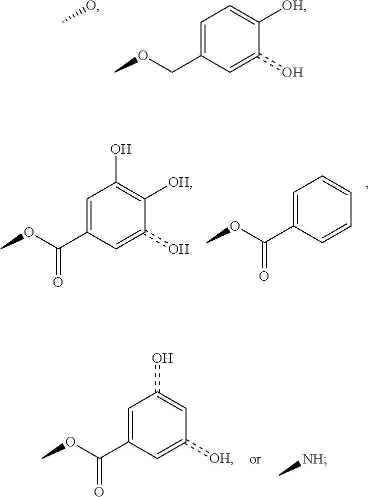

—R⁴ is —CH₃; and
—R⁵ is

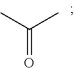

or
(e) —R¹ is

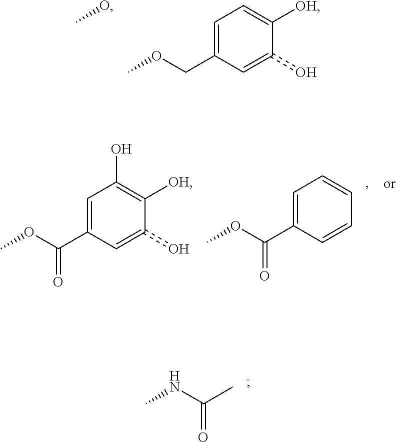

—R² is

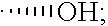

—R³ is

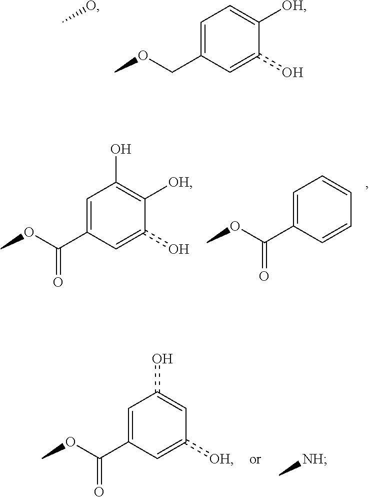

—R⁴ is —OH; and
—R⁵ is —H;

In some embodiments, compounds that bind STAT3 linker domain with affinity equal to or greater than Striatal B comprise compounds of Table 1.

TABLE 1
Compounds having affinity for STAT3 linker domain equal to or greater than Striatal B.

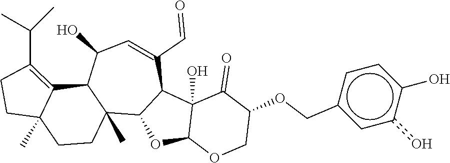

compound 1
(2aR,5bR,6S,8aR,8bS,10R,12aS,13aS,13bR)-10-((3,4-dihydroxybenzyl)oxy)-6,8b-dihydroxy-5-
isopropyl-2a,13b-dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-8-carbaldehyde

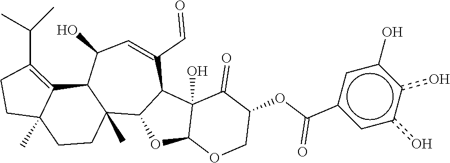

compound 2
(2aR,5bR,6S,8aR,8bS,10R,12aS,13aS,13bR)-8-formyl-6,8b-dihydroxy-5-isopropyl-2a,13b-
dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl 3,4,5-trihydroxybenzoate

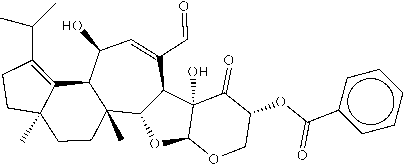

compound 3
(2aR,5bR,6S,8aR,8bS,10R,12aS,13aS,13bR)-8-formyl-6,8b-dihydroxy-5-isopropyl-2a,13b-
dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl benzoate

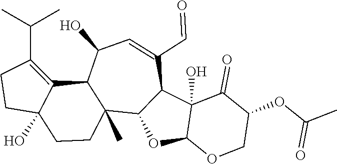 [SMILES: CC(=O)O[C@@H]1CO[C@H]2O[C@H]3[C@@H](C(C=O)=C[C@H](O)[C@@H]4C5=C(C(C)C)CC[C@]5(O)CC[C@@]34C)[C@@]2(O)C1=O]

compound 4
(2aS,5bS,6S,8aR,8bS,10R,12aS,13aS,13bR)-8-formyl-2a,6,8b-trihydroxy-5-isopropyl-13b-
methyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl acetate

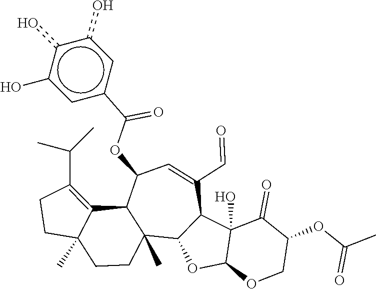

compound 5
(2aR,5bR,6S,8aR,8bS,10R,12aS,13aS,13bR)-10-acetoxy-8-formyl-8b-hydroxy-5-isopropyl-
2a,13b-dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-6-yl 3,4,5-trihydroxybenzoate

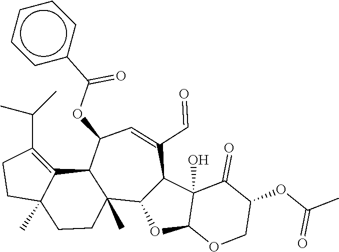

compound 6
(2aR,5bR,6S,8aR,8bS,10R,12aS,13aS,13bR)-10-acetoxy-8-formyl-8b-hydroxy-5-isopropyl-
2a,13b-dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-6-yl benzoate

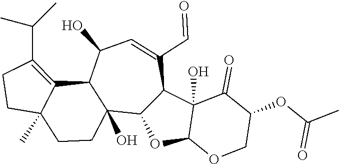 [SMILES: CC(=O)O[C@@H]1CO[C@H]2O[C@H]3[C@@H](C(C=O)=C[C@H](O)[C@@H]4C5=C(C(C)C)CC[C@]5(C)CC[C@@]34O)[C@@]2(O)C1=O]

compound 7
(2aR,5bS,6S,8aR,8bS,10R,12aS,13aS,13bS)-8-formyl-6,8b,13b-trihydroxy-5-isopropyl-2a-
methyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl acetate

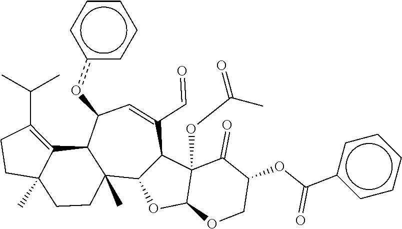

compound 8
(2aR,5bR,6S,8aR,8bS,10R,12aS,13aS,13bR)-8b-acetoxy-8-formyl-5-isopropyl-2a,13b-
dimethyl-9-oxo-6-phenoxy-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl benzoate

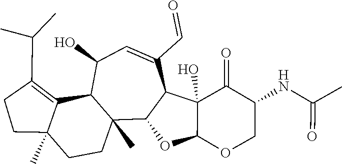 [SMILES: CC(=O)N[C@@H]1CO[C@H]2O[C@H]3[C@@H](C(C=O)=C[C@H](O)[C@@H]4C5=C(C(C)C)CC[C@]5(C)CC[C@@]34C)[C@@]2(O)C1=O]

compound 9
N-((2aR,5bR,6S,8aR,8bS,10R,12aS,13aS,13bR)-8-formyl-6,8b-dihydroxy-5-isopropyl-2a,13b-
dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl)acetamide

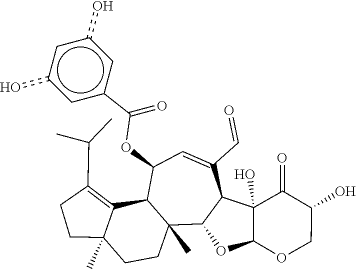

compound 10
(2aR,5bR,6S,8aR,8bS,10R,12aS,13aS,13bR)-8-formyl-8b,10-dihydroxy-5-isopropyl-2a,13b-
dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-6-yl 3,5-dihydroxybenzoate

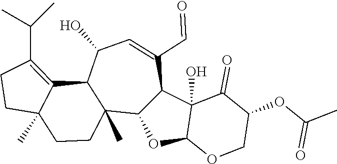 [SMILES: CC(=O)O[C@@H]1CO[C@H]2O[C@H]3[C@@H](C(C=O)=C[C@@H](O)[C@@H]4C5=C(C(C)C)CC[C@]5(C)CC[C@@]34C)[C@@]2(O)C1=O]

compound 11
(2aR,5bR,6R,8aR,8bS,10R,12aS,13aS,13bR)-8-formyl-6,8b-dihydroxy-5-isopropyl-2a,13b-
dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl acetate

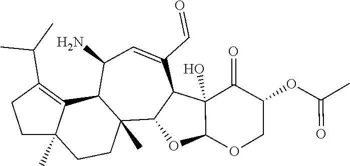 [SMILES: CC(=O)O[C@@H]1CO[C@H]2O[C@H]3[C@@H](C(C=O)=C[C@H](N)[C@@H]4C5=C(C(C)C)CC[C@]5(C)CC[C@@]34C)[C@@]2(O)C1=O]

compound 12
(2aR,5bR,6S,8aR,8bS,10R,12aS,13aS,13bR)-6-amino-8-formyl-8b-hydroxy-5-isopropyl-
2a,13b-dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl acetate

In some embodiments, the described variables and compounds of formula I or II can be a single tautomer or a mixture of tautomers. As a non-limiting example, in some embodiments, when R¹ is

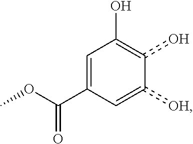

then a compound of formula I or II is a single tautomer, wherein R¹ is

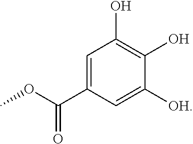

In some embodiments, when R¹ is

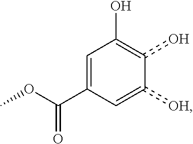

a compound of formula I or II is a single tautomer, wherein R¹ is

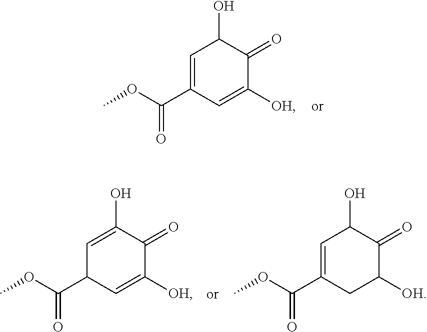

In some embodiments, when R¹ is

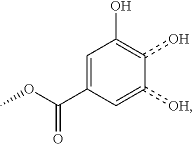

a compound of formula I or II is a mixture of two or more tautomers, wherein R¹ is

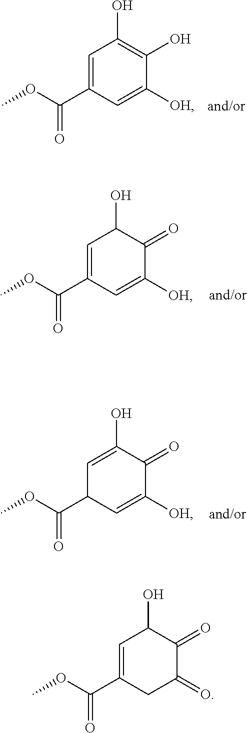

In some embodiments, compound 1, compound 2, compound 5, compound 8, and/or compound 10 can be a single tautomer or a mixture of tautomers. In some embodiments, compound 1, compound 2, compound 5, compound 8, and/or compound 10 is a single tautomer with the structures:

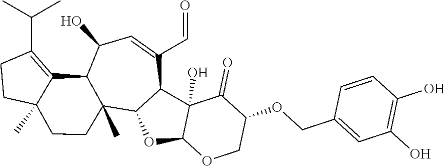 [SMILES: CC(C)C1=C2[C@H]3[C@@H](O)C=C(C=O)[C@@H]4[C@H](O[C@@H]5OC[C@@H](OCc6ccc(O)c(O)c6)C(=O)[C@@]54O)[C@]3(C)CC[C@@]2(C)CC1]

compound 1
(2aR,5bR,6S,8aR,8bS,10R,12aS,13aS,13bR)-10-((3,4-dihydroxybenzyl)oxy)-6,8b-
dihydroxy-5-isopropyl-2a,13b-dimethyl-9-oxo-
1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-8-carbaldehyde

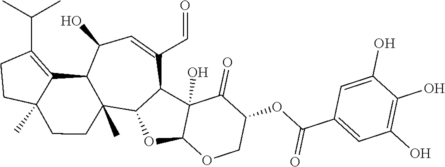 [SMILES: CC(C)C1=C2[C@H]3[C@@H](O)C=C(C=O)[C@@H]4[C@H](O[C@@H]5OC[C@@H](OC(=O)c6cc(O)c(O)c(O)c6)C(=O)[C@@]54O)[C@]3(C)CC[C@@]2(C)CC1]

compound 2
(2aR,5bR,6S,8aR,8bS,10R,12aS,13aS,13bR)-8-formyl-6,8b-dihydroxy-5-isopropyl-
2a,13b-dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-10-yl 3,4,5-
trihydroxybenzoate

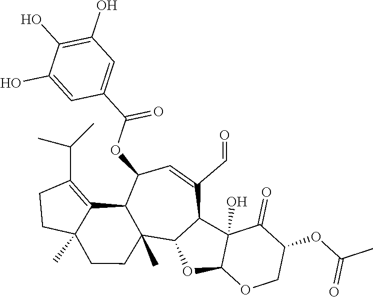 [SMILES: CC(=O)O[C@@H]1CO[C@H]2O[C@H]3[C@@H](C(C=O)=C[C@H](OC(=O)c4cc(O)c(O)c(O)c4)[C@@H]4C5=C(C(C)C)CC[C@]5(C)CC[C@@]34C)[C@@]2(O)C1=O]

compound 5
(2aR,5bR,6S,8aR,8bS,10R,12aS,13aS,13bR)-10-acetoxy-8-formyl-8b-hydroxy-5-
isopropyl-2a,13b-dimethyl-9-oxo-1,2,2a,3,4,5b,6,8a,8b,10,11,12a,13a,13b-
tetradecahydro-9H-indeno[5″,4″:3′,4′]cyclohepta[1′,2′:4,5]furo[2,3-b]pyran-6-yl 3,4,5-
trihydroxybenzoate

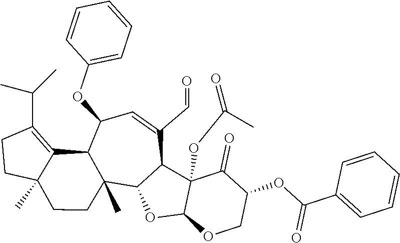 [SMILES: CC(=O)O[C@]12C(=O)[C@H](OC(=O)c3ccccc3)CO[C@H]1O[C@H]1[C@H]2C(C=O)=C[C@H](Oc2ccccc2)[C@@H]2C3=C(C(C)C)CC[C@]3(C)CC[C@@]12C]

compound 8
(2aR,5bR,6S,8aR,8bS,10R,12aS,13aS,13bR)-8b-acetoxy-8-formyl-5-isopropyl-
2a,13b-dimethyl-9-oxo-6-phenoxy-1,2,2a,3,4,5b,6,8a,8b,10,11,12a,13a,13b-
tetradecahydro-9H-indeno[5″,4″:3′,4′]cyclohepta[1′,2′:4,5]furo[2,3-b]pyran-10-yl
benzoate

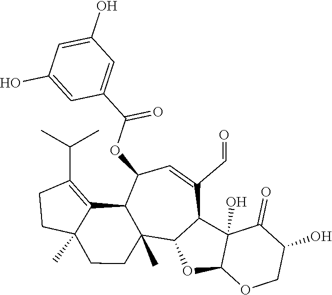 [SMILES: CC(C)C1=C2[C@H]3[C@@H](OC(=O)c4cc(O)cc(O)c4)C=C(C=O)[C@@H]4[C@H](O[C@@H]5OC[C@@H](O)C(=O)[C@@]54O)[C@]3(C)CC[C@@]2(C)CC1]

compound 10
(2aR,5bR,6S,8aR,8bS,10R,12aS,13aS,13bR)-8-formyl-8b,10-dihydroxy-5-isopropyl-
2a,13b-dimethyl-9-oxo-1,2,2a,3,4,5b,6,8a,8b,10,11,12a,13a,13b-tetradecahydro-9H-
indeno[5″,4″:3′,4′]cyclohepta[1′,2′:4,5]furo[2,3-b]pyran-6-yl 3,5-dihydroxybenzoate

In some embodiments, the described compounds of formula I or II have anti-pancreatic cancer activity.

In some embodiments, the compounds of formula I or II are formulated for use in combination with an additional anti-pancreatic therapeutic. The additional anti-pancreatic therapeutic can be, but is not limited to, gemcitabine. In some embodiments, the compounds of formula I or II are formulated for use in combination with gemcitabine.

In some embodiments, the compounds of formula I or II are formulated in combination with an additional anti-pancreatic therapeutic. The additional anti-pancreatic therapeutic can be, but is not limited to, gemcitabine. In some embodiments, the compounds of formula I or II are formulated in combination with gemcitabine.

In some embodiments, the compounds of formula I or II act synergistically in combination with gemcitabine in treating pancreatic cancer, inhibiting pancreatic cancer cell growth, and/or inhibiting communication between pancreatic cancer cells and pancreatic stellate cells.

In some embodiments, the compounds of formula I or II potentiate gemcitabine activity in a synergistic fashion.

### III. Formulations

Any of the described compounds of formula I or II can be provided in or formulated in a pharmaceutical composition. A pharmaceutical composition (or medicament) includes a pharmaceutically effective amount of at least one of the described compounds of formula I or IH and optionally one or more pharmaceutically acceptable excipients and optionally one or more additional anti-cancer therapeutics and/or additional active pharmaceutical ingredients. Pharmaceutically acceptable excipients (excipients) are substances other than the Active Pharmaceutical ingredient (API, therapeutic product (e.g., compounds of formula I or II)) that are intentionally included in the pharmaceutical composition. Excipients do not exert or are not intended to exert a therapeutic effect at the intended dosage. Excipients may act to (a) aid in processing of the API during manufacture, (b) protect, support, or enhance stability, bioavailability, or patient acceptability of the API, (c) assist in product identification, and/or (d) enhance any other attribute of the overall safety, effectiveness, or delivery of the API during storage or use. A pharmaceutically acceptable excipient may or may not be an inert substance. In some embodiments, a pharmaceutically acceptable excipient may be an inert substance. In some embodiments, a pharmaceutically acceptable excipient may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, saline, phosphate buffered saline, Ringer's solution, ethanol, polyol (e.g., glycerol, propylene glycol, and liquid polyethylene glycol), and suitable mixtures thereof. A carrier may also contain adjuvants or additives such as preservatives, wetting agents, emulsifying agents, and dispersing agents. A carrier may also contain isotonic agents, such as sugars, polyalcohols, sodium chloride, and the like.

The pharmaceutical compositions can contain other additional components commonly found in pharmaceutical compositions. Such additional components can include, but are not limited to, anti-pruritics, astringents, local anesthetics, or anti-inflammatory agents (e.g., antihistamine, diphenhydramine, etc.).

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical compositions further comprise one or more additional active ingredients. The additional active pharmaceutical ingredient can be, but is not limited to, a cancer therapeutic, a small molecule drug, an antibody, an antibody fragment, an anti-inflammatory agent, or a pain reliever. Exemplary cancer therapeutics include, but are not limited to, gemcitabine, 5-fluorouracil, irinotecan, oxaliplatin, cisplatin, capecitabine, paclitaxel, nab-paclitaxel, docetaxel, and KRAS inhibitors. KRAS inhibitors include, but are not limited to, adagrasib and sotorasib.

A compound of formula I or II or pharmaceutical composition containing a compound of formula I or II can be formulated as a liquid formulation or as a solid formulation (including a powder or lyophilized formulation; e.g., a lyophilized cake or powder). In some embodiments, compound of formula I or II or pharmaceutical composition containing a compound of formula I or II can be formulated as a tablet, a pill, a dragee, a capsule, an emulsion, a suspension, a liquid, a gel, a syrup, a slurry, a powder, or the like.

In some embodiments, a compound of formula I or II or a pharmaceutical composition containing a compound of formula I or II as described herein is formulated for administration to a subject. In some embodiments, the subject is a human subject.

### IV. Kits

The described compounds of formula I or II and the pharmaceutical compositions comprising the compounds of formula I or II disclosed herein may be packaged or included in a kit, container, pack, or dispenser. In some embodiments, the compounds of formula I or II or the pharmaceutical compositions comprising the compounds of formula I or II may be packaged in a pre-filled syringe, a vial, a pill (or similar, e.g., tablet or capsule). In some embodiments, any of the compounds of formula I or II or any of the pharmaceutical compositions comprising the compounds of formula I or II can be formulated or packaged in single-dose or multi-dose format. Any of the compounds of formula I or II or any of the pharmaceutical compositions comprising the compounds of formula I or II described herein can be formulated for repeat dosing.

The kit or package may further contain instructions for use. Instructions include documents describing relevant materials or methodologies pertaining to the kit. The instructions may include one or more of background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

### V. Delivery

The described compounds of formula I or II or the compositions containing the compounds of formula I or II described herein can be delivered to a cell, group of cells, tissue, or subject using methods known in the art.

Any of the compounds of formula I or II or any of the compositions containing the compounds of formula I or II can be formulated for oral administration, injection, infusion, intravascular administration, intravenous administration, intradermal administration, subcutaneous administration, intramuscular administration, or intraperitoneal administration.

A route of administration is the path by which a drug (e.g., compound of formula I or II) is brought into contact with the body. In general, methods of administering drugs and nucleic acids for treatment of a mammal are well known in the art and can be applied to administration of the described compounds and compositions. The described compounds and compositions can be administered via any suitable route in a preparation appropriately tailored to that route. In general, any suitable method recognized in the art for delivering small molecule drug can be adapted for use with a herein described compounds and compositions. Routes of administration include, but are not limited to, oral, parenteral, local, direct injection, intraparenchymal, intratumoral, intramuscular, implantation, topical, systemic, intravascular, intravenous, intra-arterial, intracutaneous, subdermal, subcutaneous, and intraperitoneal.

### VI. Methods of Treatment

The compounds of formula I or II and the compositions containing the compounds of formula I or II described herein can be used in the treatment or pancreatic cancer or the preparation of a medicament for use in the treatment of pancreatic cancer.

The compounds of formula I or II and the compositions containing the compounds of formula I or II described herein can be used to inhibit pancreatic cancer cell growth and/or inhibiting communication between pancreatic cancer cells and pancreatic stellate cells. The compounds of formula I or II and the compositions containing the compounds of formula I or II described herein can be used in the preparation of a medicament for use in inhibiting pancreatic cancer cell growth, and/or inhibiting communication between pancreatic cancer cells and pancreatic stellate cells.

In some embodiments, methods of treating a subject having a pancreatic cancer are described, the methods comprising administering to the subject a therapeutically effective amount of a compound of formula I or II or a composition containing a compound of formula I or II. The subject can be, but is not limited to, a human subject. In some embodiments, the subject has been previously identified or diagnosed as having a pancreatic cancer.

In some embodiments, methods are described for treating pancreatic cancer in a subject comprising administering to the subject any of the described compounds of formula I or II or a pharmaceutical composition containing any of the described compounds of formula I or II. Treating pancreatic cancer can include, but is not limited to, treating pancreatic ductal adenocarcinoma or treating metastatic pancreatic cancer. In some embodiments, the compound is selected from the group consisting of: compound 1, compound 2, compound 3, compound 4, compound 5, compound 6, compound 7, compound 8, compound 9, compound 10, compound 11, and compound 12. In some embodiments, the compound is administered in combination with gemcitabine.

In some embodiments, methods are described for inhibiting pancreatic cancer cell growth in a subject comprising administering to the subject any of the described compounds of formula I or II or a pharmaceutical composition containing any of the described compounds of formula I or II. Inhibiting pancreatic cancer cell growth can include, but is not limited to inhibiting the growth of pancreatic cancer cells or pancreatic stellate cells. In some embodiments, the compound is selected from the group consisting of: compound 1, compound 2, compound 3, compound 4, compound 5, compound 6, compound 7, compound 8, compound 9, compound 10, compound 11, and compound 12. In some embodiments, the compound is administered in combination with gemcitabine.

In some embodiments, methods are described for inhibiting communication between pancreatic cancer cells and pancreatic stellate cells in a subject comprising administering to the subject any of the described compounds of formula I or II or a pharmaceutical composition containing any of the described compounds of formula I or II. In some embodiments, the compound is selected from the group consisting of: compound 1, compound 2, compound 3, compound 4, compound 5, compound 6, compound 7, compound 8, compound 9, compound 10, compound 11, and compound 12. In some embodiments, the compound is administered in combination with gemcitabine.

In some embodiments, a compound of formula I or II or a composition containing a compound of formula I or II is administered to a subject to treat pancreatic cancer, reduce the growth of pancreatic cancer, or delay progression of pancreatic cancer.

In some embodiments, a compound of formula I or II or a composition containing a compound of formula I or II is administered to a subject having pancreatic cancer to prolong survival of the subject as compared to expected survival in the absence of treatment.

In some embodiments, a compound of formula I or II or a composition containing a compound of formula I or II is administered to a subject to decrease one or more of symptoms of with pancreatic cancer.

In some embodiments, a compound of formula I or II or a composition containing a compound of formula I or II is administered to a subject to decrease the severity of one or more symptoms of pancreatic cancer.

In some embodiments, a compound of formula I or II or a composition containing a compound of formula I or II is administered to a subject to decrease the adverse effect of one or more symptoms of pancreatic cancer.

In some embodiments, a compound of formula I or II or a composition containing a compound of formula I or II is administered to a subject to decrease the pathological consequences of pancreatic cancer.

In some embodiments, a compound of formula I or II or a composition containing a compound of formula I or II is administered to a subject to delay progression of pancreatic cancer or delay onset of one or more symptoms or pathological consequence of pancreatic cancer.

In some embodiments, a compound of formula I or II or a composition containing a compound of formula I or II is administered to a subject to prevent or partially prevent the occurrence or recurrence of pancreatic cancer, a symptom or pancreatic cancer, or a pathological consequence or pancreatic cancer.

The compounds of formula I or II or the compositions containing the compounds of formula I or II for use in the described methods are administered to a subject in an amount that is sufficient to achieve the desired therapeutic effect in the subject. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors, including the route of administration. Dosage can also depend on the duration of the treatment, age, gender, weight, condition, general health, and prior medical history of the subject being treated.

Treating a subject with a compound of formula I or II or a composition containing a compound of formula I or II can optionally be combined with administering one or more additional (e.g., second, third, etc.) therapeutics or treatments. In some embodiments, the additional therapeutic is another cancer therapeutic. In some embodiments, the one or more additional therapeutics or treatments comprises one or more of the gemcitabine, 5-fluorouracil, irinotecan, oxaliplatin, cisplatin, capecitabine, paclitaxel, nab-paclitaxel, docetaxel, and KRAS inhibitors. KRAS inhibitors include, but are not limited to, adagrasib and sotorasib. In some embodiments, the additional therapeutic comprises gemcitabine.

The compound of formula t or II and the one or more additional therapeutics can be combined in a single dosage form or pharmaceutical composition or the compound of formula I or II and the one or more additional therapeutics may be provided in separate single dosage forms or pharmaceutical compositions. The compound of formula I or II and the one or more additional therapeutics can be administered in a single composition or they may be administered separately, as in a combination therapy. The compound of formula I or II, with or without the one or more additional therapeutics, can be combined with one or more excipients to form pharmaceutical compositions.

All patent filings, websites, other publications, accession numbers and the like cited above or below are incorporated by reference in their entirety for all purposes to the same extent as if each individual item were specifically and individually indicated to be so incorporated by reference. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1. Computational Docking and Methods

Computational docking was used to identify compounds with increased binding to STAT3 relative to palmatine. From an initial screen of 139,735, Striatal B was identified with superior binding to STAT3. Using in vitro Striatal B was found to significantly inhibit pancreatic cancer cell growth and potentiate gemcitabine effects. Striatal B also reduced levels of STAT3 and epithelial-mesenchymal transition markers, indicating a mechanism of action that involves altering cell mechanics.

A. Molecular docking to STAT3: To prepare the STAT3 structure for docking, all water molecules were removed, and the residues were protonated using Chimera (PMID: 15264254). The molecular surface was analyzed for potential binding pockets using a sphere-based approach. Docking sites were identified with the SPHGEN program, which creates a grid reflecting the geometric features of each site, and these were further refined using the CLUSTER program. This program groups spheres to pinpoint the docking points for the DOCK software, version 5.1.0 (PMID: 11394736), aligning potential ligand atoms with these spheres. The DOCK algorithm incorporates intermolecular AMBER energy scoring (combining van der Waals and electrostatic interactions), contact scoring, and bump filtering. Atomic coordinates for 139,735 small molecules from the NIH Developmental Therapeutics Program's library of drug-like compounds were positioned into the most promising structural pocket, as determined by Fpocket. Each molecule was tested in 1,000 different orientations, evaluating their interactions based on polar (hydrogen bond) and nonpolar (van der Waals) forces. The best orientations and interaction scores (contact and electrostatic) were computed. Visual representations of these interactions were created using PyMOL 3.1. The 40 compounds (Table 2) ranked highest for STAT3 binding were obtained from National Cancer Institute Chemotherapeutic Agents Repository.

TABLE 2
Top 40 compounds that bound to STAT3.

| Compound | Reference number |
|---|---|
| 1 | 18082-M/I |
| 2 | 18092-W/1 |
| 3 | 18093-X/1 |
| 4 | 22627-A/I |
| 5 | 28403-G/1 |
| 6 | 40664-I/1 |
| 7 | 46715-K/1 |
| 8 | 62571-T/I |
| 9 | 67309-T/I |
| 10 | 71709-A/I |
| 11 | 75745-0/2 |
| 12 | 77799-V/I |
| 13 | 89446-H/1 |
| 14 | 92765-0/2 |
| 15 | 95007-Z/3 |
| 16 | 99721-Y/I |
| 17 | 101743-X/I |
| 18 | 120578-V/2 |
| 19 | 120582-ZT3 |
| 20 | 123232-H/1 |
| 21 | 126703-F/1 |
| 22 | 134576-M/1 |
| 23 | 225035-M/1 |
| 24 | 225045-W/l |
| 25 | 298886-K/I |
| 26 | 312033-Y/2 |
| 27 | 313172-N/1 |
| 28 | 340049-A/1 |
| 29 | 365107-0/2 |
| 30 | 378693-H/1 |
| 31 | 382454-T/1 |
| 32 | 403969-G/3 |
| 33 | 408954-X/1 |
| 34 | 409736-X/2 |
| 35 | 608344-A/1 |
| 36 | 628443-X/1 |
| 37 | 63 6356-Y/1 |
| 38 | 64 1816-KIl |
| 39 | 657315-HIl |
| 40 | 66642 5-J/l |

B. Cell lines: Human pancreatic cancer cell lines MIA PaCa-2, PANC-1, BxPC-3, and ASPC-1 were purchased from American Type Culture Collection (ATCC). MIA PaCa-2 cells were grown in DMEM media supplemented with 10% fetal bovine serum (FBS) and 5% horse serum. PANC-1, BxPC-3, Capan-2 cells were grown in DMEM, RPMI, McCoy's 5a media, supplemented with 10% fetal bovine serum (FBS) respectively. All culture media contain 1% Antibiotic-Antimycotic (100×) (Gibco, Thermo Fisher Scientific, Waltham, MA).

C. Cell viability assay: In initial screening, cells were exposed to the compounds for 48 h at a final concentration of 1 to 25 μM. Logarithmically growing pancreatic cancer cells were seeded at a density of 4000 cells/100 μL complete media per well in 96-well plates in triplicates. After attachment (~24 h), cells were treated with different concentrations of compounds (1-25 μM) for an additional 48 h. Cell viability was determined by CellTiter 96® Non-Radioactive Cell Proliferation Assay (MTT Assay) (Promega, Madison, WI). Based on these data, we selected compounds showing 50% growth inhibition at concentrations lower than 10 M for additional dose response studies. Subsequent dose response (0.25 to 10 M) studies were conducted in multiple pancreatic cancer cell lines essentially as described above. In combination studies, escalating concentrations of both Striatal B and gemcitabine were used in pancreatic tumor organoids. First, we determined IC₅₀ concentration for individual drugs (Striatal B and gemcitabine) in the organoid models. Subsequently, we used 4×IC₅₀, 2×IC₅₀, IC₅₀, 1/2×IC₅₀, 1/4×IC₅₀ for both compounds in combination studies. Bliss independence and Loewe additivity models were used to determine whether the combinations exerted synergistic or additive effect as described in Vlot A H C et al. 2019 (“Applying synergy metrics to combination screening data: agreements, disagreements and pitfalls.” Drug Discov Today. 2019; 24(12):2286-2298). A score greater than 5 indicated synergy, and a score, less than −5 indicated antagonism for both models. Synergy scores based on Bliss independence suggested that 2 drugs elicited their responses independently by targeting separate pathways. On the other hand, Loewe additivity model indicated that both drugs had similar modes of action on the same targets or pathways.

D. Colony formation assay: Logarithmically growing PANC-1, ASPC-1, hPSCs, and mouse KPC cells plated in 2 mL complete media in 6-well plates were treated with Striatal B for 48 h. Following treatment, media was replaced with fresh media with no drug. Cells were maintained for 10-14 days for colonies to form. Colonies were stained with 0.1% crystal violet in methanol and counted as described in Franken N A et al. 2006 (“Clonogenic assay of cells in vitro.” Nat Protoc. 2006; 1(5):2315-2319).

E. Human pancreatic organoid lines: Four different pancreatic organoid lines including PA13004B, PA15055B, PA2410B, and PA5389B were used. Organoid lines were maintained and cultured in advanced DMEM/F12 (12634028 Life Technologies; or DMEM medium (Hyclone SH30243.01). Briefly 40 μL suspension containing 250 organoids in cell culture media mixed with 50% matrigel in a 1:1 ratio (final concentration of 5% v/v) were seeded in triplicate in 384 well plates. Escalating concentrations of Striatal B (0, 0.0015, 0.0046, 0.0137, 0.041, 0.123, 0.370,1.111, 3.333 and 10 μM) was added to the organoid suspension and plates were incubated at 37° C. for 5 days. Proliferation was determined by using CellTiter-Glo 3D cell viability assay essentially as described by the manufacturer. Cell survival was calculated relative to vehicle control. Dose response curve was fitted using non-linear regression model with a sigmoidal dose response to calculate IC₅₀ using Graph Pad PRISM 7.0.

F. CyTOF analysis: Single-cell suspension from ASPC-1 and PANC-1 cells were stained with cisplatin for discrimination of live cells from dead cells. Cells were then fixed in 1.6% paraformaldehyde and stained with a panel of metal-conjugated antibodies (Table 3). The cells were then stained with Cell-ID Intercalator-Ir (191Ir and 193Ir) (Fluidigm, South San Francisco, CA) for identification of nucleated cells during CyTOF analysis. After staining, cells were loaded onto Helios mass cytometer with EQ Four Element Calibration Beads (Fluidigm, South San Francisco, CA) for signal normalization. Signals of samples were normalized using CyTOF software (Version 6.7.1014, Fluidigm). For analysis in ASPC-1 and PANC-1 cells, the generated files underwent signal cleanup and gating for live cells using Cytobank. Based on expression level of 140 Ce (cesium) and 191 Ir (iridium), the singlet cells (140 Ce⁻/191 Ir⁺) were gated from ungated cells. Within singlet cells, the live cells (195 Pt⁻/191 Ir⁺; Pt=cisplatin) were gated based on expression level of 195 Pt and 191 Ir. Subpopulations of cells from samples were clustered using PhenoGraph clustering algorithm embedded in cytofkit2 and visualized using t-distributed stochastic neighbor embedding (t-SNE) algorithm based on expression levels of 17 markers (hepatocyte growth factor receptor (c-MET), zinc finger E-box-binding homeobox 2 (ZEB2), N-Cadherin, Slug, zinc finger E-box-binding homeobox 1 (ZEB1), Janus kinase 1 (JAK1), Fibronectin, Vimentin, pSTAT3, phosphorylated extracellular signal-regulated kinase 1 (pERK1), AXL receptor tyrosine kinase (AXL), β-catenin, Snail, TWISTI, Zonula occludens-1 (ZO-1), epithelial cell adhesion molecule (EpCAM), and SRY-Box transcription factor 9 (SOX9)). Heat maps showing expression level among clusters were generated using MeV 4.9.0 based on median expression level of each protein in each cluster. Violin plots were generated based on pSTAT3 expression level in each cluster by R package ggplot2 based on arcsine-transformed CyTOF data from cytofkit2. A heat map of clusters with normalized expression level of each protein were aligned with increasing levels of pSTAT3. The percentage of each cluster (subpopulation) in each treatment group was calculated and cluster subpopulation plots were generated by R package ggplot2. To explore intratumor cell heterogeneity, the identified subpopulations were classified based on pSTAT3 levels into three subgroups (I: pSTAT3-low, II: pSTAT3-intermediate and III: pSTAT3-high).

TABLE 3
Metal-conjugated antibodies

| | Antigen | Metal tag |
|---|---|---|
| 1 | SNAI1 | 166Er |
| 2 | SOX9 | 175Lu |
| 3 | B-catenin | 165Ho |
| 4 | P-Erk 1/P-Erk 2 | 160Gd |
| 5 | CD31/PECAM | 144Nd |
| 6 | AXL1 | 161Dy |
| 7 | N-Cadherin | 143Nd |
| 8 | Twist1 | 167Er |
| 9 | ZO-1 | 169Tm |
| 10 | ITGA5 | 170Er |
| 11 | ZEB1 | 151Eu |
| 12 | EpCAM | 173Yb |
| 13 | VEGF | 150Nd |
| 14 | CD45 | 176Yb |
| 15 | EGFR/ErbB1 | 159Tb |
| 16 | ZEB2 | 142Nd |
| 17 | Slug | 149Sm |
| 18 | Vimentin | 156Gd |
| 19 | Arginase 1 | 164Dy |
| 20 | CD44 | 171Yb |
| 21 | CD273/PD-L2 | 172Yb |
| 22 | YAP1 | 209Bi |
| 23 | Ki-67 | 162Dy |
| 24 | COL4A1 | 152Sm |
| 25 | COL5A2 | 145Nd |
| 26 | c-MET | 141Pr |
| 27 | FN1 | 155Gd |
| 28 | TAZ | 181Ta |
| 29 | JAK1 | 153Eu |
| 30 | pSTAT3 | 158Gd |
| 31 | PD-1/CD279 | 174Yb |

G. Immunoblot analysis: Whole-cell extracts were prepared using 2×sodium dodecyl sulfate (SDS) lysis buffer with protease inhibitor and phosphate inhibitor and heated at 99° C. for 10 min. Lysates were separated on polyacrylamide gels containing SDS before transferred on nitrocellulose membranes. The blots were probed with indicated primary antibodies at 4° C. overnight, and appropriate secondary antibody at room temperature for 1 h. The blots were developed with Western Lightning Plus ECL chemiluminescent reagents (Perkin Elmer, Waltham, MA) and detected with Syngene G: Box imaging system.

H. Atomic Force Microscopy: Atomic Force Microscopy was performed as described in Clark A et al. 2024 (“Targeting S6K/NFκB/SQSTM1/Polθ signaling to suppress radiation resistance in prostate cancer.” Cancer Lett. 2024; 597:217063).

I. Statistical analysis: Data are presented as mean±SEM. Statistical analysis for (i) gene expression changes (delat Cq in PANC-1 and ASPC-1) was performed by mixed effects model (REML) using Sidak's multiple comparisons test; and (ii) colony formation assays using one way ANOVA followed by Dunnett's T3 multiple comparison test using GraphPad Prism software. Statistical significance is determined by P<0.05 (*P<0.05, **P<0.01, ***P<0.001, ****P<0.0001).

### Example 2. Identification of Striatal B

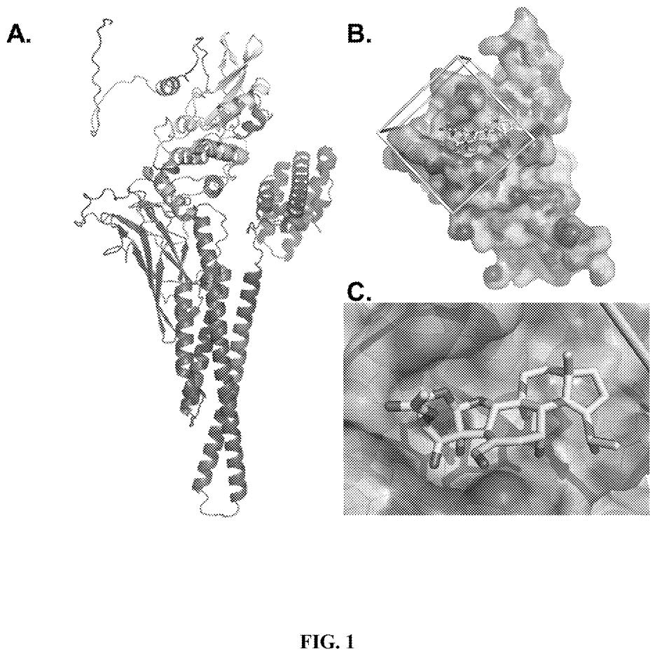

The interaction site between palmatine and STAT3 was investigated to identify structural elements involved in its activity in cancer. The complete STAT3 structure, predicted by AlphaFold, was used to assess druggability. Druggability assessment with Fpocket revealed that a structural pocket within the linker domain (positions 466-579), adjacent to the SH2 domain (580-670), was highly likely to be druggable (FIG. 1A). Palmatine exhibited a predicted higher affinity for this linker domain site compared to other STAT3 regions. This site was then used as a basis for selecting drug-like molecules capable of binding to the STAT3 linker domain. Out of 139,735 compounds screened, the top 40 scoring compounds were selected for validation assays. These assays identified Striatal B as the most effective compound in modulating STAT3 activity. Striatal B possesses “drug-like” physical and chemical properties that adhere to the Lipinski's rule of five (FIGS. 1B and 1C), which include a molecular mass under 488.6 Daltons, a topological polar surface area (tPSA) of 119.4, and an optimal number of hydrogen bond donors (2) and acceptors (7). Additionally, its charged nature reduces its logP value to 1, indicating favorable solubility characteristics (Lipinski C A. “Lead- and drug-like compounds: the rule-of-five revolution.” Drug Discov Today Technol. 2004; 1(4):337-341).

### Example 3. Striatal B Inhibits Growth of Cell Culture Models of PDAC

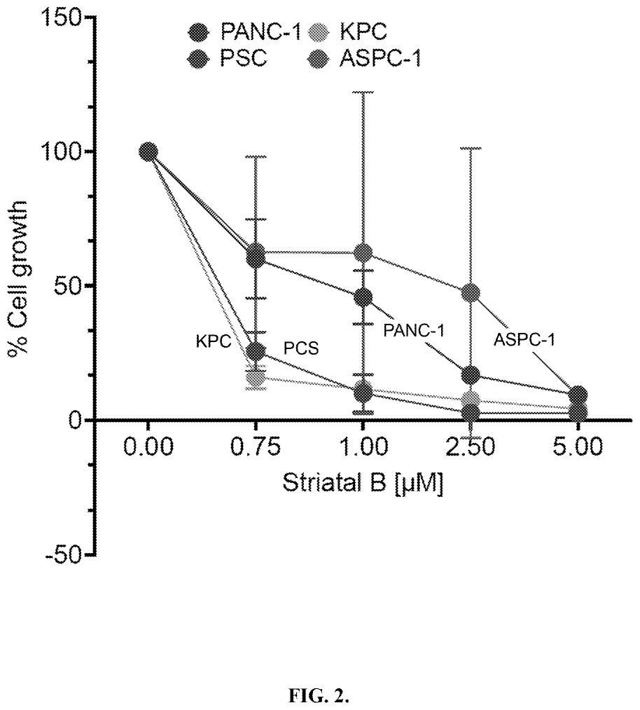

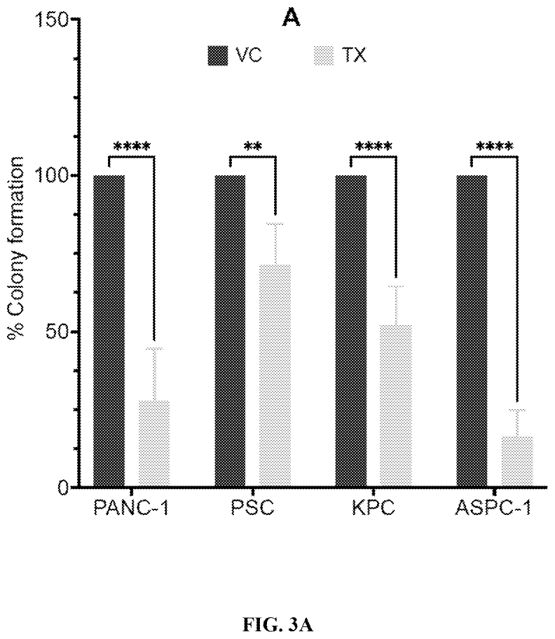

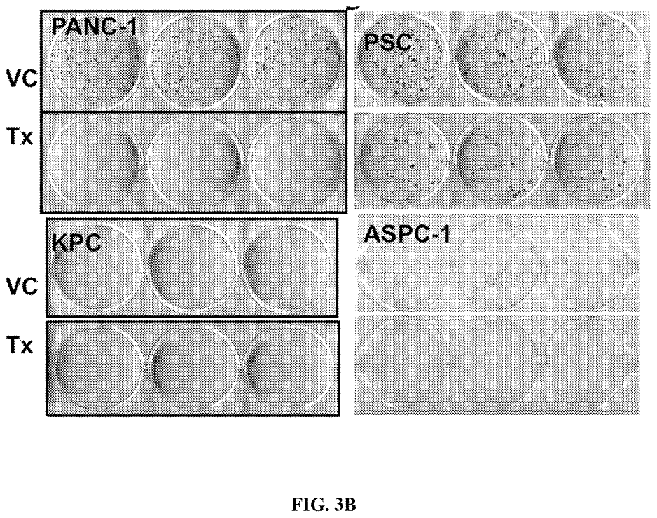

The inhibitory effects of Striatal B on the growth of pancreatic cancer cell lines were assessed using cell viability and colony forming ability as outcomes. We observed 50% inhibition in viability at low micromolar concentrations of Striatal B in human PDAC cell lines, activated human pancreatic stellate cells (hPSCs), and mouse pancreatic cancer cell line (FIG. 2). We validated these results using in vitro clonogenicity to determine the ability of Striatal B to inhibit the formation of colonies as described in Franken N A et al. 2006. As shown in FIG. 3, Striatal B decreased the colony forming ability of PANC-1,ASPC-1, hPSCs, and mKPC cells significantly.

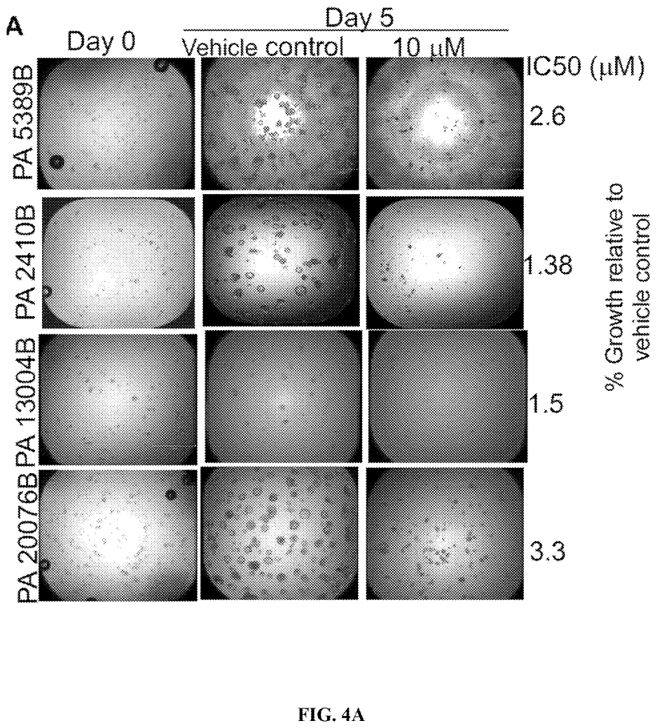

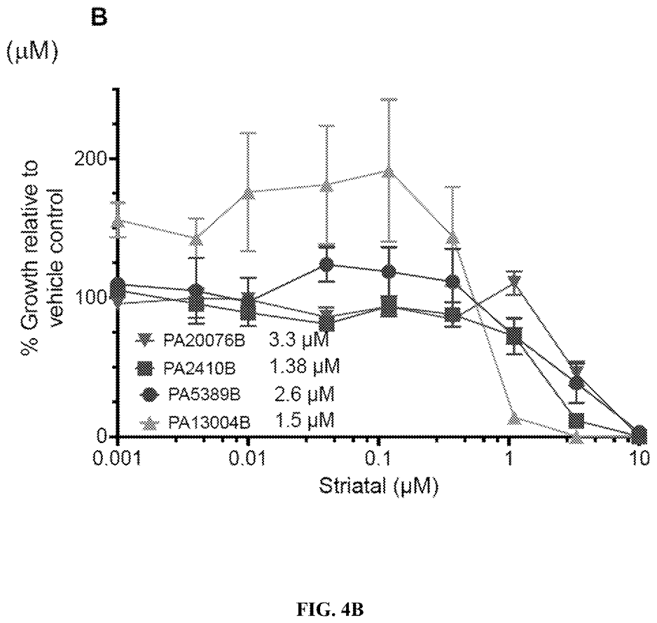

Next, we tested the effect of Striatal B on the growth of human pancreatic cancer organoid lines developed by Crown Bio. FIG. 4A shows the size of organoids at day 0 and day 5 in the presence of Striatal B relative to vehicle control. Analyses of these data show that all four models used were sensitive to Striatal B at low micromolar concentrations (FIG. 4A-B).

### Example 4. Synergistic Effects of Striatal B and Gemcitabine Combination

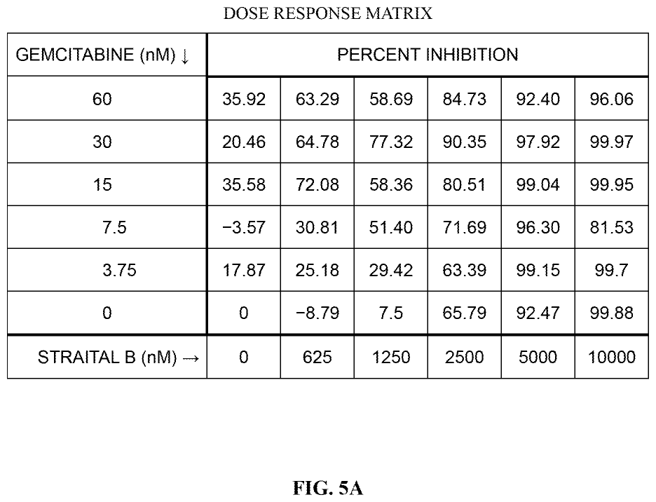

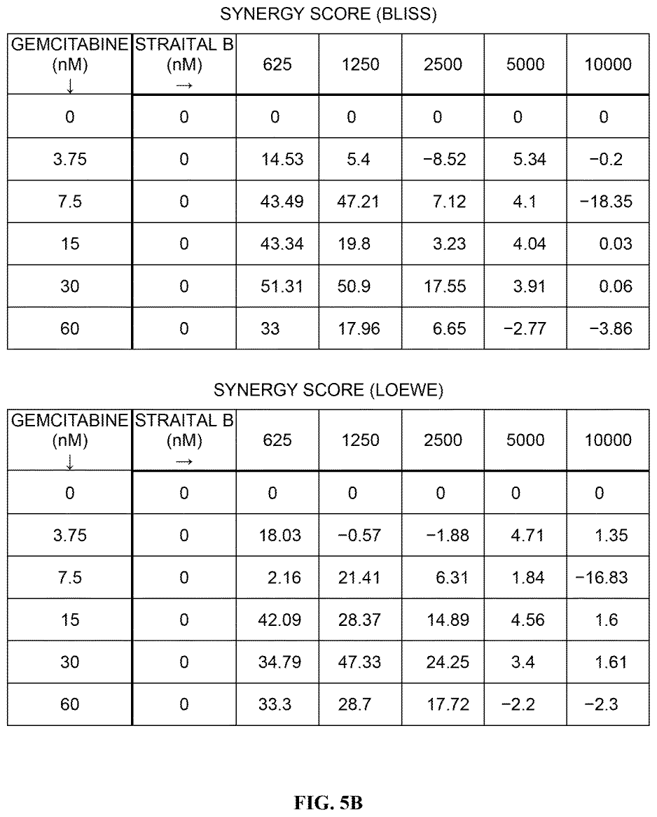

Since gemcitabine is associated with the least adverse events compared with the current regimens used our goal we determined if combining gemcitabine with Striatal B could improve therapeutic potential. We tested the potential utility of Striatal B in combination with gemcitabine in pancreatic tumor organoids. A heat map showing response to Striatal B and gemcitabine singly and in combination over a range of concentrations used is shown (FIG. 5A). Further analysis of these data revealed that, for most of the concentrations tested, the combination showed synergy based on both Bliss and Loewe models (FIG. 5B). These observations suggest that there is potential combinatorial benefits of the combination of Striatal B and gemcitabine in human pancreatic cancer organoid models.

### Example 5. Striatal B Decreases STAT3-AXL Signaling in PDAC Cells and Mechanical Properties Associated with Aggressiveness

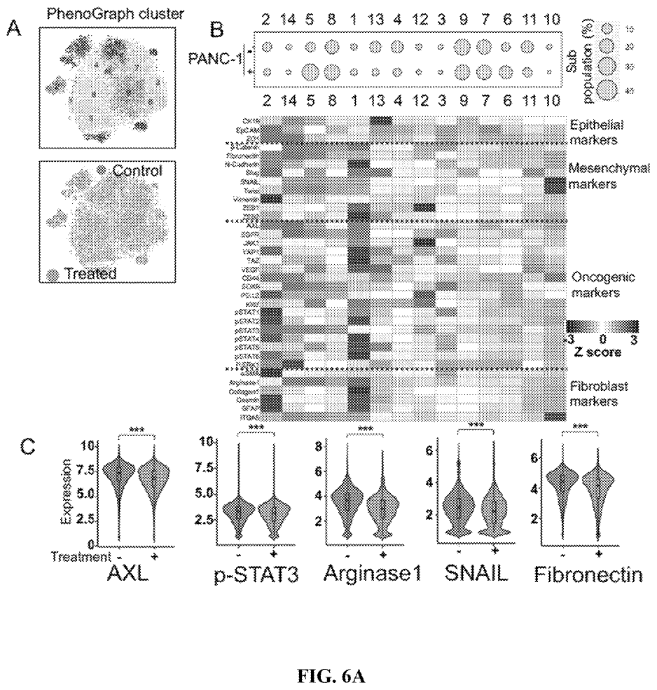

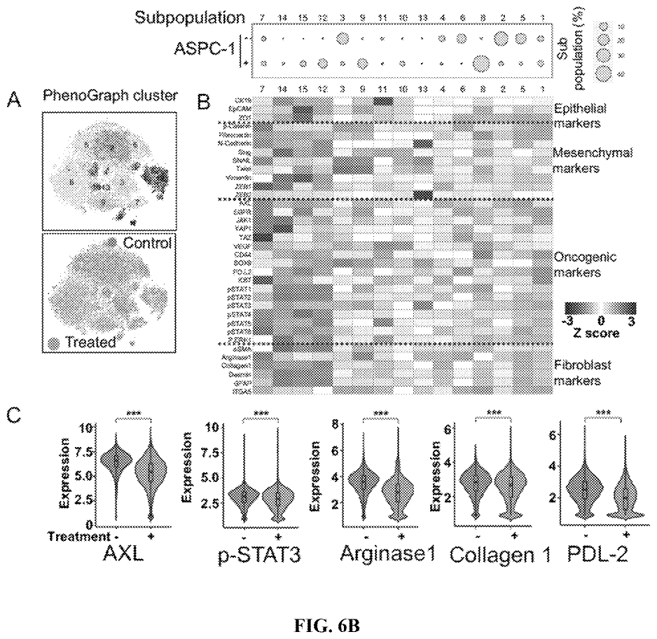

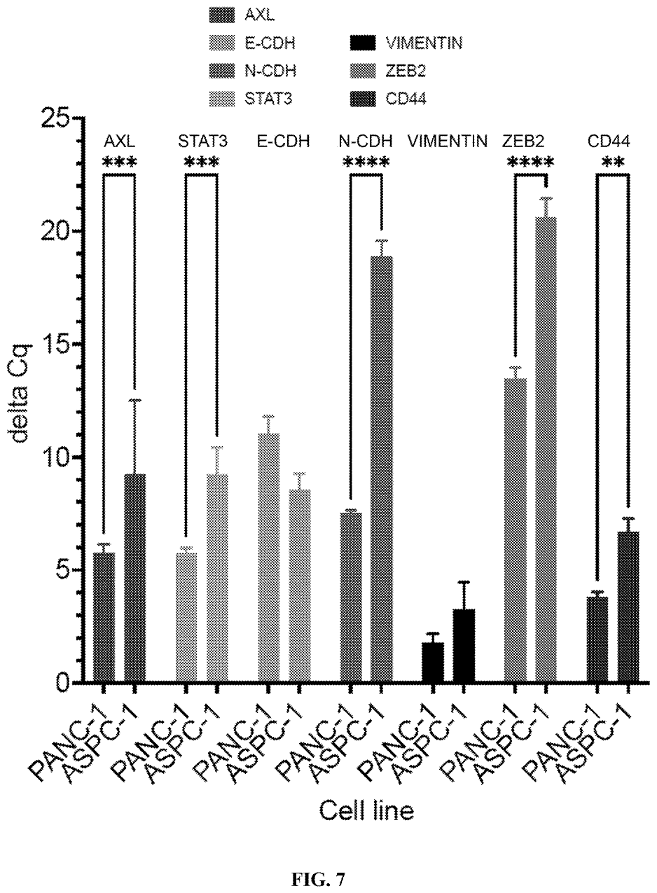

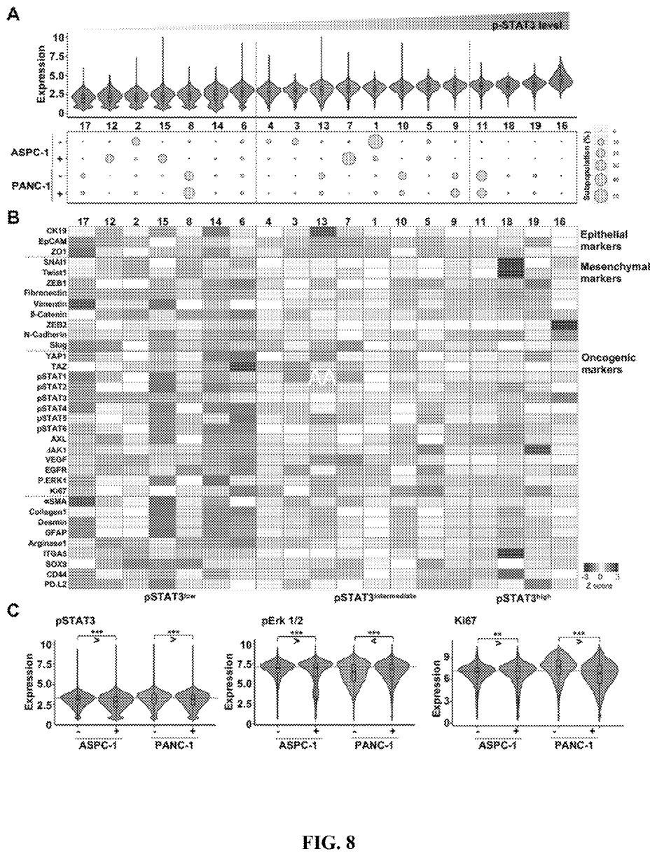

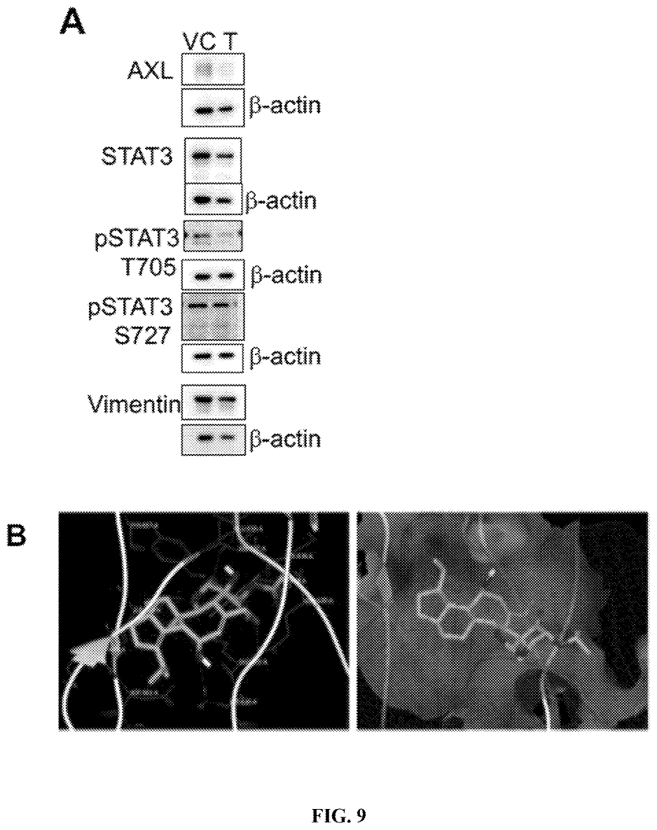

We used CyTOF to gain insight into signaling changes that occur in response to treatment with Striatal B. Following treatment with Striatal B, PANC-1, and ASPC-1 cells (with low and high sensitivity to Striatal B) were stained with metal-conjugated antibodies that recognize markers associated with epithelial mesenchymal transition (EMT), and oncogenic signaling. 60,000 EpCAM+ single cells were categorized into 14 and 15 sub-populations respectively in PANC-1 and ASPC-1 based on PhenoGraph algorithm for clustering in tSNE plots. Interestingly, although these sub-populations showed different levels of protein expression, Striatal B treatment markedly decreased expression of oncogenic and EMT markers, albeit differently, between the 2 cell lines (FIGS. 6A and 6B). For example, Ki67, AXL, EGFR, pSTAT2, pSTAT3, pSTAT5, pSTAT6, collagen 1, GFAP, Arginase 1, and EMT markers such as β-catenin, N-cadherin, vimentin, fibronectin, Snail, ZEB2, and Slug decreased significantly following treatment in PANC-1 cells (FIG. 6A). On the other hand, ASPC-1 cells showed marked reduction in AXL, pSTAT2, pSTAT3, pSTAT4, pSTAT5, pSTAT6, Arginase 1, and PDL2 while changes in EMT markers were less prominent (FIG. 6B). Notably qPCR data shows that the ASPC-1 cell line is more epithelial in nature compared with PANC-1 cells based on the difference in gene expression between these two cell lines (FIG. 7). Given the commonality between the 2 cells with respect to STAT proteins and the previously demonstrated involvement of STAT3 signaling in PDAC, we aligned these clusters based on pSTAT3 levels. These data suggest that most of the oncogenic markers correlated positively with STAT3 in both cell lines (FIG. 8). These observations indicate that Striatal B inhibits STAT3 results in in downregulation of oncogenic signaling pathways. Consistent with these observations, although treatment had no significant effect on mRNA expression of STAT3 or other EMT markers, Striatal B reduced STAT3 activation as revealed by decreased levels of pSTAT3 and AXL in pancreatic cancer cells (FIG. 9A). Furthermore, using computational docking with FRED.1BG1.pdb as a template, we found that STAT3 interacts with Striatal B. We observed two hydrogen bonding interactions within the SH2 domain and Striatal B. The aldehyde group of Striatal B makes hydrogen bond with Gln637 and oxygen atom present in the six membered ring (pyrone-in Striatal B) makes hydrogen bond with Glu638. We also observed hydrophobic effects with Phe716, Pro715, Thr714, Ile659, Tyr657 and Trp623 (FIG. 9B).

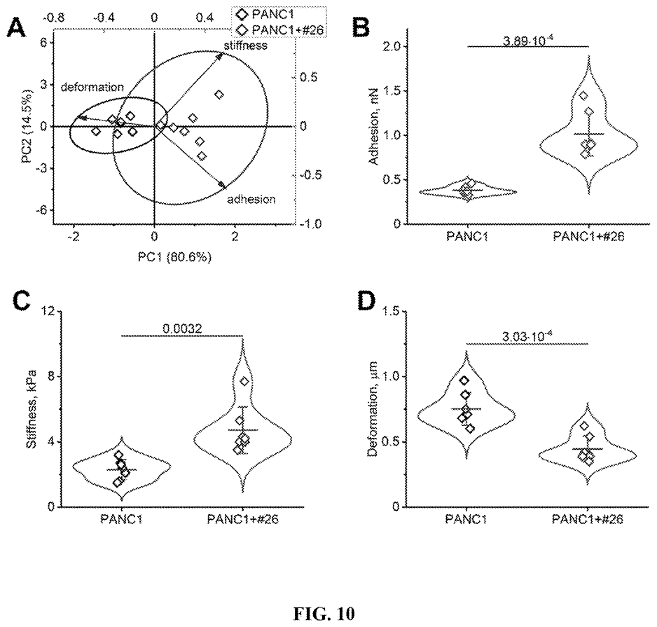

Interestingly, in atomic force microscopy studies, Striatal B treatment displayed increased stiffness, decreased deformation, and decreased adhesion suggesting that it alters cellular fitness, which in turn decreases the aggressive nature of cancer cells. Consistent with these observations, principal component analysis clearly separated populations of PANC-1 cells treated with Striatal B from untreated counterparts (FIG. 10). Taken together, these observations show that Striatal B perturbs oncogenic signaling to reduce the aggressiveness of PDAC cells.

STAT3 represents a promising target for management of cancer. Previous studies identified the SH2 domain as amenable to binding bioactive compounds Sgrignani J et al “Structural biology of STAT3 and its implications for anticancer therapies development.” International Journal of Molecular Sciences, 2018 19(6); Coleman D R et al “Investigation of the binding determinants of phosphopeptides targeted to the SRC homology 2 domain of the signal transducer and activator of transcription 3. development of a high-affinity peptide inhibitor.” Journal of Medicinal Chemistry, 2005 48(21):6661-6670; Ren Z et al. “Identification of a high-affinity phosphopeptide inhibitor of Stat3.” Bioorganic & Medicinal Chemistry Letters, 2003 13(4), 633-636; and Turkson J et al. “Phosphotyrosyl peptides block Stat3-mediated DNA binding activity, gene regulation, and cell transformation.” The Journal of Biological Chemistry, 2001 276(48):45443-45455). Surprisingly, we now show the linker domain of STAT3 is a suitable target for cancer drug development. Our findings show that this region, spanning amino acids 466 to 583 and lying adjacent to the SH2 domain, possesses high druggability according to assessments using Fpocket. Furthermore, the successful binding of palmatine to this domain, and the subsequent identification of Striatal B as a potent modulator of STAT3 activity, underscore the therapeutic potential of targeting this part of the protein. These results challenge the prevailing focus on traditional binding sites.

We demonstrated that Striatal B inhibits the growth of pancreatic cancer cells and human pancreatic cancer organoids at low micromolar concentrations. Computational docking studies further reveal that Striatal B interacts with STAT3.

Striatal B represents a novel compound in cancer treatment, including pancreatic ductal adenocarcinoma (PDAC). Our results demonstrate that Striatal B not only reduces growth and colony-forming ability of multiple pancreatic cancer cell lines but also affects stellate cells in the PDAC tumor microenvironment. Additionally, Striatal B enhances the growth-inhibitory effects of gemcitabine in human pancreatic cancer organoids. Striatal B also reduced phosphorylated STAT3 (pSTAT3) levels and decreased markers of epithelial-mesenchymal transition (EMT) in PANC-1 cells. Striatal B also appears to alter the mechanical properties of pancreatic cancer cells, thereby reducing their metastatic potential.

### Example 6. Design of STAT3 Binding Molecules

21 new chemical entities were designed, based on the Striatal B, and screened for STAT3 linker domain binding by molecular docking. 12 of the 21 new chemical entities (shown in Table 1) were predicted to bind STAT3 with equal to or higher affinity relative to Striatal B (Table 4).

TABLE 4
Molecular docking calculated binding affinity of designed
compounds for binding to STAT3 linker domain.

| Compound | ΔG |
|---|---|
| Compound 1 | −7.9 |
| Compound 2 | −7.8 |
| Compound 3 | −7.7 |
| Compound 4 | −7.3 |
| Compound 5 | −7.2 |
| Compound 6 | −7.2 |
| Compound 7 | −7.1 |
| Compound 8 | −7.1 |
| Compound 9 | −7.1 |
| Compound 10 | −7.0 |
| Compound 11 | −7.0 |
| Compound 12 | −6.9 |
| Striatal B | −6.9 |

These compounds are evaluated for their ability to inhibit the proliferation of both human and mouse pancreatic cancer cells and tumor organoids and to synergize with gemcitabine as described above for Striatal B. The compounds of Table 1 are predicted to have increase affinity for and increased anti-pancreatic cancer activity relative to Striatal B.

1. A compound having the structure of formula II: 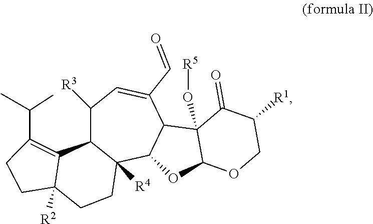
wherein:
—R¹ is 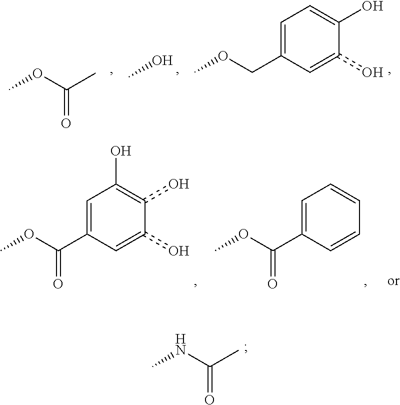
—R² is 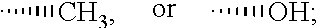
—R³ is 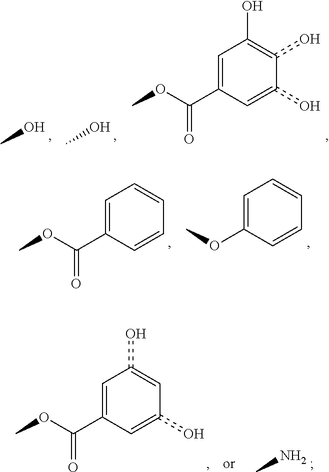
—R⁴ is —CH₃ or —OH; and
—R⁵ is —H or 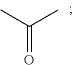
and
wherein:
(a) if —R¹ is 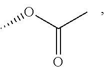
then
—R² is 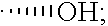
—R³ 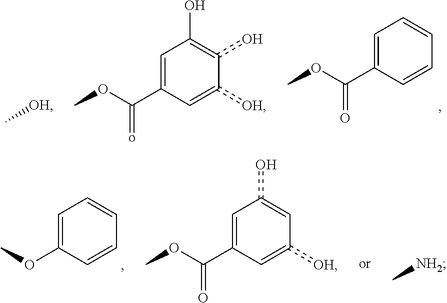
—R⁴ —OH; and/or
—R⁵ 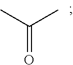
(b) if —R² is 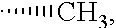
then
—R¹ is 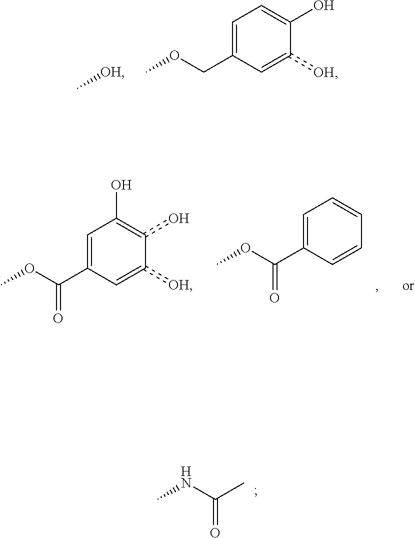
—R³ is, 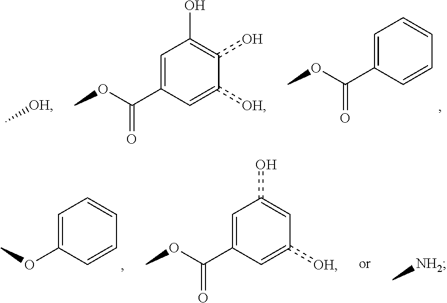
—R⁴ —OH; and/or
—R⁵ is 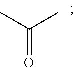
(c) if —R³ is 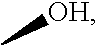
then
—R¹ is 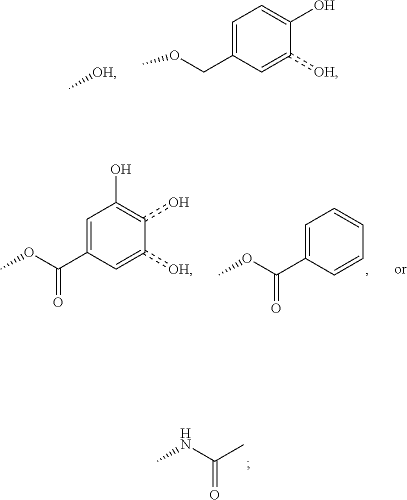
—R² is 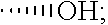
—R⁴ is —OH; and/or
—R⁵ is 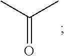
(d) if —R⁴ is —CH₃, then
—R¹ is 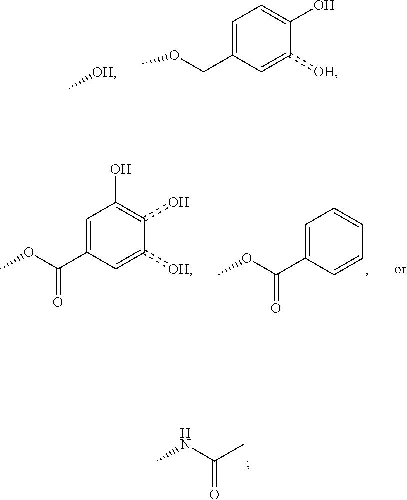
—R² is 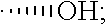
—R³ is 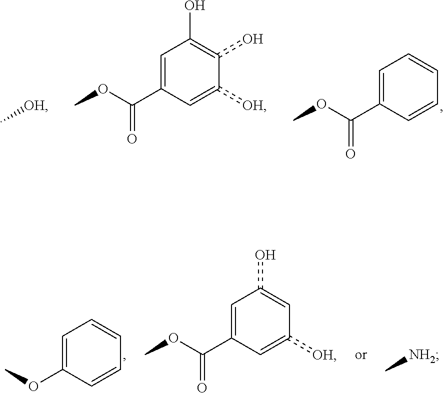
and/or
—R⁵ is 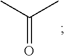
and
(e) if —R⁵ is —H; then
—R¹ is 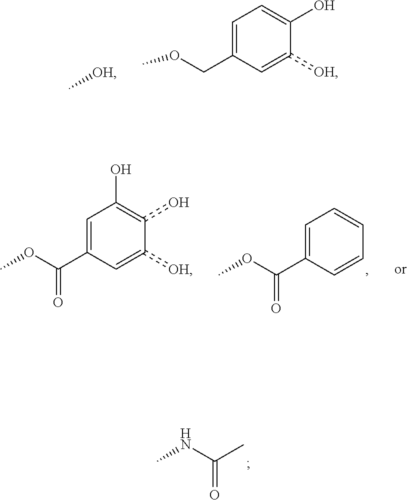
—R² is 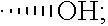
—R³ is 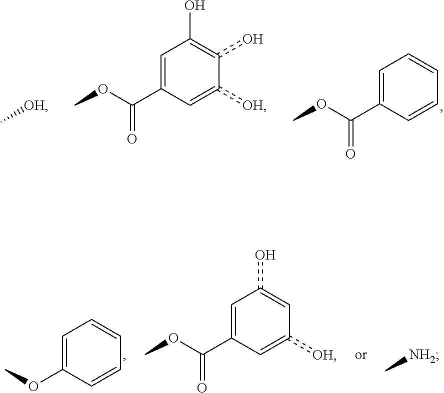
and/or
—R⁴ is —OH.

2. The compound of claim 1, wherein:
—R¹ is

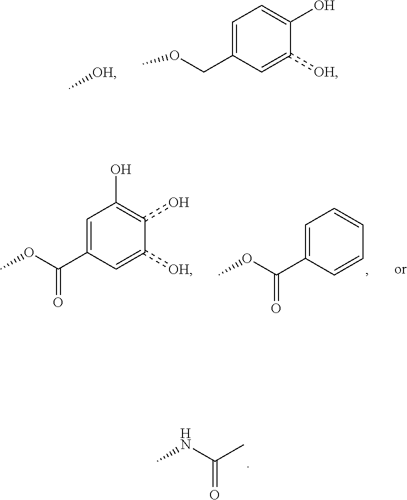

3. The compound of claim 1, wherein:
—R³ is

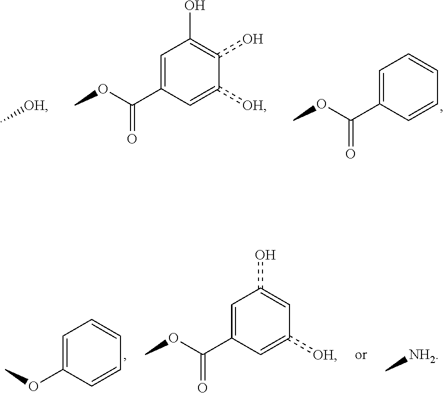

4. The compound of claim 1, wherein: —R⁴ is —OH.

5. The compound of claim 1, wherein: —R⁵ is

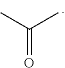

6. The compound of claim 1, wherein:
—R¹ is

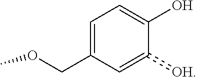

7. The compound of claim 1, wherein:
—R¹ is

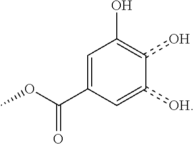

8. The compound of claim 1, wherein:
—R¹ is

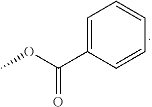

9. The compound of claim 1, wherein: —R² is

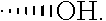

10. The compound of claim 1, wherein:
—R³ is

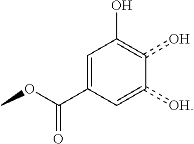

11. The compound of claim 1, wherein:
—R³ is

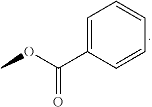

12. The compound of claim 1, wherein —R⁴ is —OH.

13. The compound of claim 1, wherein:
—R¹ is 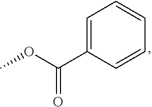
—R³ is 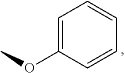
and —R⁵ is

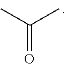

14. The compound of claim 1, wherein:
—R¹ is

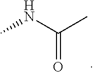

15. The compound of claim 1, wherein:
—R¹ is 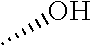
and —R³ is

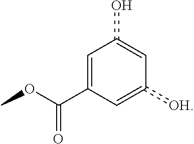

16. The compound of claim 1, wherein: —R³ is

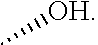

17. The compound of claim 1, wherein:—R³ is

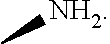

18. The compound of claim 1, wherein the compound is selected from the group consisting of:

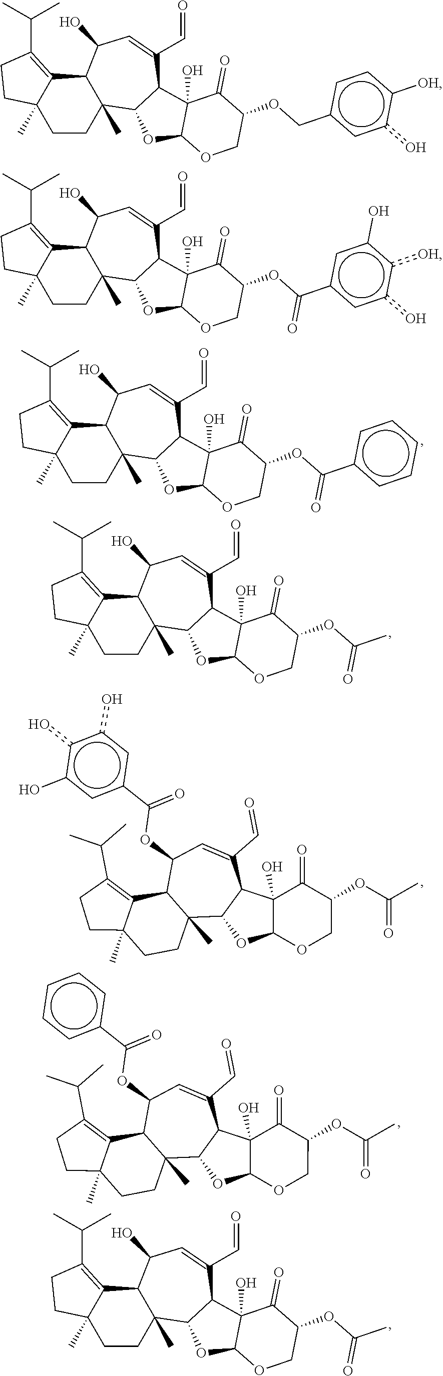

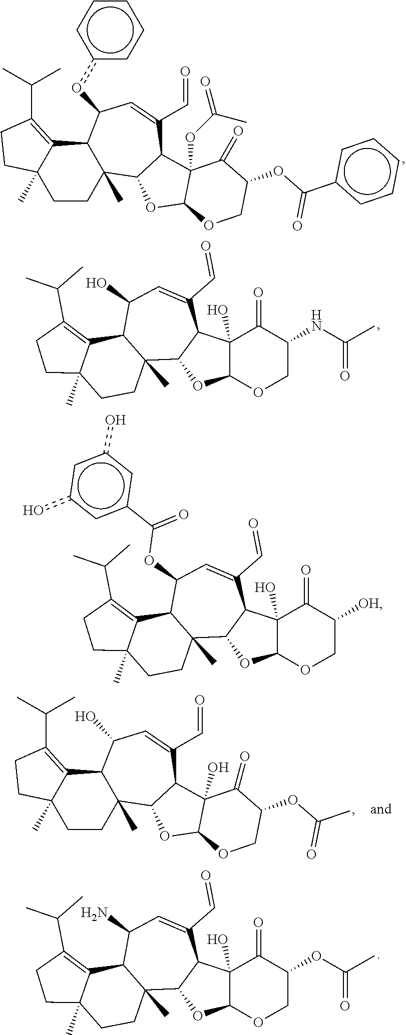

19. A method of treating pancreatic cancer in a subject comprising: administering to the subject a therapeutically effective dose of the compound of claim 1.
